(19) United States
(12) Patent Application Publication
(10) Pub. No.: US 2023/0346759 A1
(43) Pub. Date: Nov. 2, 2023

(54) NOVEL THERAPEUTIC USES OF COMPOUNDS FOR ENHANCING MITOCHONDRIAL FUNCTION AND TREATING MITOCHONDRIAL DISEASES

(71) Applicants: STANDIGM INC., Seoul (KR); THE CHILDREN'S HOSPITAL OF PHILADELPHIA, Philadelphia, PA (US)

(72) Inventors: Minyoung SO, Ann Arbor, MI (US); Jinhan KIM, Seoul (KR); Sang Ok SONG, Seongnam-si, Gyeonggi-do (KR); Sanghyung JIN, Seoul (KR); Marni FALK, Voorhees, NJ (US); Heeyong YOON, Havertown, PA (US); Bhumi SHAH, Dayton, NJ (US); Eiko OGISO, Havertown, PA (US)

(21) Appl. No.: 18/007,407

(22) PCT Filed: Jul. 28, 2021
(86) PCT No.: PCT/KR2021/009824

§ 371 Date: Jan. 30, 2023

Related U.S. Application Data
(60) Provisional application No. 63/057,992, filed on Jul. 29, 2020.

(51) Int. Cl. A61K 31/4422; A61K 31/7048; A61K 31/573; A61K 31/519; A61K 31/444

(52) U.S. Cl. CPC A61K 31/4422; A61K 31/7048; A61K 31/573; A61K 31/519; A61K 31/444

(57) ABSTRACT

Novel therapeutic uses of compounds for enhancing mitochondrial function and treating a mitochondrial disease are discovered by artificial intelligence (AI)-based in silico approaches and validated by in vitro and in vivo animal model studies in mitochondrial disease models. Methods of enhancing mitochondrial function and/or treating mitochondrial disease include administering an active compound to a subject in need.

### CROSS REFERENCE TO RELATED APPLICATIONS

This application claims the benefit of and priority to U.S. Provisional Application No. 63/057,992 filed Jul. 29, 2020, the content of which is incorporated herein by reference in its entirety.

### TECHNICAL FIELD

The present technology relates generally to novel therapeutic uses of compounds for enhancing mitochondrial function and treating mitochondrial diseases.

### BACKGROUND

Mitochondrion is an organelle present in most eukaryotic cells, of which primary function is oxidative phosphorylation, a process through which energy derived from metabolism of glucose or fatty acids is converted to adenosine triphosphate (ATP). ATP is then used to drive various energy-requiring biosynthetic reactions and other metabolic activities. In addition to generating cellular ATP, mitochondria are also involved in other cellular functions, such as cellular homeostasis, signaling pathways, and steroid synthesis. Diseases arising from mitochondrial dysfunction may include for example, but not limited to, mitochondrial swelling due to mitochondrial membrane potential malfunction, functional diseases due to oxidative stress such as by the action of reactive oxygen species (ROS) or free radicals, functional diseases due to genetic mutations and diseases due to functional deficiency of oxidative phosphorylation mechanisms for energy production.

Mitochondria deteriorate with age, losing respiratory activity, accumulating damage to their DNA (mtDNA) and producing excessive amounts of ROS.

The mechanism of mitochondrial diseases (or pathological conditions associated with mitochondrial dysfunctions) has not been clearly understood until today. Recent evidence points to involvement of mitochondrial dysfunction in several diseases, for example, Leber hereditary optic neuropathy (LHON), mitochondrial myopathy, encephalopathy, lactic acidosis and stroke-like episodes (MELAS) syndrome, mitochondrial complex I deficiency, mitochondrial complex II deficiency, mitochondrial complex III deficiency, mitochondrial complex IV deficiency, mitochondrial complex V deficiency, Leigh syndrome, autosomal dominant optic atrophy (ADOA), leukoencephalopathy with brain stem and spinal cord involvement and lactate elevation (LBSL), Luft disease, multiple acyl-CoA dehydrogenase (MAD) deficiency, mitochondrial enoyl CoA reductase proteinassociated neurodegeneration (MEPAN) syndrome, mitochondrial DNA depletion, mitochondrial encephalopathy, pyruvate carboxylase deficiency, mitochondrial myopathy, Friedreich's ataxia, Barth syndrome, fatal infantile cardioencephalomyopathy, CharcotMarie-Tooth disease, infantile lactic acidosis, congenital lactic acidosis (CLA), chronic lactic acidosis, Kearns-Sayre syndrome (KSS), mitochondrially inherited diabetes and deafness (MIDD), Alpers-Huttenlocher syndrome (AHS), childhood myocerebrohepatopathy spectrum (MCHS), ataxia neuropathy spectrum (ANS; previously referred to as mitochondrial recessive ataxia syndrome (MIRAS) and sensory ataxia neuropathy dysarthria and ophthalmoplegia (SANDO)), myoclonic epilepsy myopathy sensory ataxia (MEMSA; previously referred to as spinocerebellar ataxia with epilepsy (SCAE)), Sengers syndrome, MEGDEL syndrome (also known as 3-methylglutaconic aciduria with deafness, encephalopathy and Leigh-like syndrome), Pearson syndrome, myoclonic epilepsy with ragged red fibers (MERRF), neurogenic muscle weakness, ataxia and retinitis pigmentosa (NARP), chronic progressive external ophthalmoplegia (CPEO), mitochondrial neurogastrointestinal encephalopathy (MNGIE) syndrome, carnitine deficiency, carnitine-acylcarnitine translocase (CACT) deficiency, carnitine palmitoyl transferase 1A (CPT I) deficiency, carnitine palm itoyl transferase (CPT II) deficiency, creatine deficiency syndromes, creatine deficiency syndromes which contain guanidinoacetate methyltransferase (GAMT) deficiency, L-arginine:glycine amidinotransferase (AGAT) deficiency, or creatine transporter deficiency (including SLC6A8-related creatine transporter deficiency), thymidine kinase 2 deficiency (TK2D), pyruvate dehydrogenase complex deficiency (PDCD), fatty acid oxidation disorders (FAOD), fatty acid oxidation disorders which contain acyl-CoA dehydrogenase 9 (ACAD9) deficiency, multiple acyl-CoA dehydrogenase deficiency (MADD), long-chain acyl-CoA dehydrogenase (LCAD) deficiency, long-chain 3-hydroxyacyl-CoA dehydrogenase (LCHAD) deficiency, medium-chain acyl-CoA dehydrogenase (MCAD) deficiency, short-chain acylCoA dehydrogenase (SCAD) deficiency, short-chain 3-hydroxyacyl-CoA dehydrogenase (SCHAD) deficiency or very long-chain acyl-CoA dehydrogenase (VLCAD) deficiency, co-enzyme Q10 deficiency, and multiple mitochondrial dysfunction syndrome.

Generally, treatment of a mitochondrial disease is palliative and aimed at treating symptoms and improving quality-of-life (QoL). Palliative treatments include, for example, administering vitamins, conserving energy, controlling dietary intake, and reducing stress on the body.

### BRIEF SUMMARY

The present description has the aim of providing novel therapeutic uses of compounds in treating mitochondrial diseases. The new uses may be discovered by artificial intelligence (AI)-based in silico approaches. According to the present description, the above object is achieved thanks to the subject matter specifically recalled in the ensuing claims, which are understood as forming an integral part of this disclosure. The result obtained through the present description indicates that the AI-predicted compounds have the ability to increase mitochondrial function.

The present description in some embodiments provides a method for enhancing mitochondrial function in a subject in need thereof, which comprises administering to such subject an effective amount of one or more compounds selected from the group consisting of: josamycin, cyproterone, cilnidipine, felodipine, trapidil, metyrapone, and a pharmaceutically acceptable salt, an isomer, a hydrate, and a tautomer thereof.

In some embodiments, a pharmaceutically acceptable salt of cyproterone is acetate. The present description in some embodiments provides a method for enhancing mitochondrial function, wherein enhancing mitochondrial function is, but not limited to, one or more events selected from the group consisting of: (i) enhancing cell viability; (ii) increasing intracellular ATP content; (iii) increasing mitochondrial membrane potential; (iv) reducing mitochondrial ROS; (v) reducing intracellular ROS; and (vi) reducing mitochondrial stress, compared to prior to administering the active compound described herein.

The present description in some embodiments provides a method for enhancing organismal health and survival, wherein enhancing organismal health and survival is, but not limited to, one or more events selected from the group consisting of: (i) increasing lifespan, (ii) enhancing neuronal activity; (iii) enhancing locomotor activity; and (iv) enhancing growth, compared to prior to administering the active compound described herein.

The mechanism of mitochondrial diseases has not been fully elucidated yet, however it is known that mitochondrial dysfunction is involved in the diseases. Therefore, enhancing the function of mitochondria may be effective in treating and relieving mitochondrial diseases. It also has been confirmed by the present specification that known therapeutic agents, for example phosphocreatine and RTA-408 (omaveloxolone), for mitochondrial diseases enhances mitochondrial function.

The present description in some embodiments provides a method for treating a mitochondrial disease by enhancing mitochondrial function in a subject, comprising administering to such subject an effective amount of one or more compounds selected from the group consisting of: josamycin, cyproterone, cilnidipine, felodipine, trapidil, metyrapone, and a pharmaceutically acceptable salt, an isomer, a hydrate, and a tautomer thereof. In some embodiments, a pharmaceutically acceptable salt of cyproterone is acetate.

The present description in some embodiments provides a composition for enhancing mitochondrial function in a subject in need thereof and/or for treating a mitochondrial disease by enhancing mitochondrial function in a subject in need thereof, wherein said composition comprises as an active ingredient, one or more compounds selected from the group consisting of josamycin, cyproterone, cilnidipine, felodipine, trapidil, metyrapone, and a pharmaceutically acceptable salt thereof, wherein an effective amount of the composition is administered to the subject.

In some embodiments, a pharmaceutically acceptable salt of cyproterone is acetate. The present description in some embodiments provides a composition for enhancing mitochondrial function, wherein enhancing mitochondrial function is, but not limited to, one or more events selected from the group consisting of: (i) enhancing cell viability; (ii) increasing intracellular ATP content; (iii) increasing mitochondrial membrane potential; (iv) reducing mitochondrial ROS; (v) reducing intracellular ROS; and reducing mitochondrial stress, compared to prior to administering the active compound described herein. The present description in some embodiments provides a composition for enhancing organismal health and survival, wherein enhancing organismal health and survival is, but not limited to, one or more events selected from the group consisting of: (i) increasing lifespan, (ii) enhancing neuronal activity; (iii) enhancing locomotor activity; and (iv) enhancing growth, compared to prior to administering the active compound described herein.

In some embodiments according to method or a composition described herein, the mitochondrial disease comprises, but not limited to, one or more selected from the group consisting of: Leber hereditary optic neuropathy (LHON), mitochondrial myopathy, encephalopathy, lactic acidosis and stroke-like episodes (MELAS) syndrome, mitochondrial complex I deficiency, mitochondrial complex II deficiency, mitochondrial complex III deficiency, mitochondrial complex IV deficiency, mitochondrial complex V deficiency, Leigh syndrome, autosomal dominant optic atrophy (ADOA), leukoencephalopathy with brain stem and spinal cord involvement and lactate elevation (LBSL), Luft disease, multiple acyl-CoA dehydrogenase (MAD) deficiency, mitochondrial enoyl CoA reductase protein-associated neurodegeneration (MEPAN) syndrome, mitochondrial DNA depletion, mitochondrial encephalopathy, pyruvate carboxylase deficiency, mitochondrial myopathy, Friedreich's ataxia, Barth syndrome, fatal infantile cardioencephalomyopathy, Charcot-Marie-Tooth disease, infantile lactic acidosis, congenital lactic acidosis (CLA), chronic lactic acidosis, Kearns-Sayre syndrome (KSS), mitochondrially inherited diabetes and deafness (MIDD), Alpers-Huttenlocher syndrome (AHS), childhood myocerebrohepatopathy spectrum (MCHS), ataxia neuropathy spectrum (ANS; previously referred to as mitochondrial recessive ataxia syndrome (MIRAS) and sensory ataxia neuropathy dysarthria and ophthalmoplegia (SANDO)), myoclonic epilepsy myopathy sensory ataxia (MEMSA; previously referred to as spinocerebellar ataxia with epilepsy (SCAE)), Sengers syndrome, MEGDEL syndrome (also known as 3-methylglutaconic aciduria with deafness, encephalopathy and Leigh-like syndrome), Pearson syndrome, myoclonic epilepsy with ragged red fibers (MERRF), neurogenic muscle weakness, ataxia and retinitis pigmentosa (NARP), chronic progressive external ophthalmoplegia (CPEO), mitochondrial neurogastrointestinal encephalopathy (MNGIE) syndrome, carnitine deficiency, carnitine-acylcarnitine translocase (CACT) deficiency, carnitine palmitoyl transferase 1A (CPT I) deficiency, carnitine palm itoyl transferase (CPT II) deficiency, creatine deficiency syndromes, creatine deficiency syndromes which contain guanidinoacetate methyltransferase (GAMT) deficiency, L-arginine:glycine amidinotransferase (AGAT) deficiency, or creatine transporter deficiency (including SLC6A8-related creatine transporter deficiency), thymidine kinase 2 deficiency (TK2D), pyruvate dehydrogenase complex deficiency (PDCD), fatty acid oxidation disorders (FAOD), fatty acid oxidation disorders which contain acyl-CoA dehydrogenase 9 (ACAD9) deficiency, multiple acyl-CoA dehydrogenase deficiency (MADD), long-chain acyl-CoA dehydrogenase (LCAD) deficiency, long-chain 3-hydroxyacyl-CoA dehydrogenase (LCHAD) deficiency, medium-chain acyl-CoA dehydrogenase (MCAD) deficiency, short-chain acylCoA dehydrogenase (SCAD) deficiency, short-chain 3-hydroxyacyl-CoA dehydrogenase (SCHAD) deficiency or very long-chain acyl-CoA dehydrogenase (VLCAD) deficiency, co-enzyme Q10 deficiency, and multiple mitochondrial dysfunction syndrome.

### BRIEF DESCRIPTION OF DRAWINGS

FIGS. 1(A)-1(C) show protective effects of three drug compounds, josamycin, cyproterone, and cilnidipine that have drawn through structural similarity-based approach. Quiescent SH-SY5Y cells were treated with each compound with indicated concentrations at 4 hours before 1 mM MPP⁺ (1-methyl-4-phenylpyridinium; a mitochondrial complex I inhibitor) treatment for 20 hours. A reference drug, phosphocreatine (pCr) (1 mM) was also pretreated at 4 hours before the treatment of 1 mM MPP⁺ for 20 hours. FIG. 1(A) shows intracellular ATP contents, FIG. 1(B) shows tetramethylrhodamine ethylester (TMRE)-mediated mitochondrial membrane potential, and FIG. 1(C) shows cell viability measured by methylthiazoletetrazolium (MTT) assay. All values are reported as a percentage of the control (% Control). The data are plotted as the mean±standard error of measurement (n=6) (###p<0.001 vs. DMSO-control; * p<0.05, ** p<0.01, and *** p<0.001 vs. MPP⁺-treated control. p values are from a one-way ANOVA followed by Tukey's test).

FIGS. 2(A)-2(E) show dose-dependent protective effects of five drug compounds, josamycin, cilnidipine, felodipine, trapidil, and metyrapone. Quiescent SH-SY5Y cells were treated with each compound with indicated concentrations at 4 hours before the treatment of 1 mM MPP⁺ for 20 hours. Reference drugs, phosphocreatine (pCr) (1 mM) and RTA-408 (10 nM) were also pretreated at 4 hours before the treatment of 1 mM MPP⁺ for 20 hours. FIG. 2(A) shows cell viability by MTT assay, FIG. 2(B) shows intracellular ATP content, FIG. 2(C) shows TMRE-mediated mitochondrial membrane potential, FIG. 2(D) shows mitochondrial ROS content, and FIG. 2(E) shows intracellular ROS content in a dose response manner. All values are reported as % control. The data are plotted as the mean±standard error of measurement (n=4-6) (#p<0.05 vs. DMSO-control; * p<0.05 vs. MPP⁺-treated DMSO. Statistical analyses were performed by Student's two tailed t-test).

FIGS. 3(A)-4(C) show rescue effects of trapidil (FIGS. 3(A)-3(C)) and metyrapone (FIGS. 4(A)-4(C)) through Lifespan assay. Mitochondrial complex I mutant, C. elegans gas-1 (fc21) were treated with trapidil and metyrapone at indicated concentrations.

FIGS. 3(A) and 4(A) show activity, FIGS. 3(B) and 4(B) show mortality, and FIGS. 3(C) and 4(C) show body length of C. elegans wild-type (N2) and gas-1(fc21) worms. The data are plotted as the mean±standard deviation (n=1-2 biological replicates with 30 worms per treatment condition for Experiments (A) and (B), n=5 or 10 individual worms for Experiment (C)) (*p<0.05, **p<0.01, and *** p<0.001 vs. N2 control).

FIGS. 5(A) and 5(B) show protective effects of josamycin and cilnidipine through mitochondria stress assay. L4440, untreated empty vector was used as control, and omaveloxolone was used as a reference drug. FIG. 5(A) shows josamycin's effects and FIG. 5(B) shows cilnidipine's effects. Statistical significance threshold was set at p<0.05, and with statistical analyses were performed by Student's t-test in Graphpad Prism V8 (San Diego, CA: GraphPad Software Inc.).

FIG. 6 shows rescue effects of cilnidipine through TMRE-mediated mitochondrial membrane potential. As a reference material, 25 μM of omaveloxolone was used. The date are plotted as the mean±standard error of measurement (n=minimum of 35 worms/condition). Statistical significance threshold was wet set at p<0.05, and statistical analyses were performed in Graphpad Prism. Statistical analyses were performed by Student's t-test *p<0.05, **p<0.01, and ***p<0.001.

FIGS. 7(A)-7(E) show beneficial effects of josamycin, cyproterone, felodipine, trapidil, and metyrapone through efficacy test in mitochondrial complex IV disease model. Wild type, AB zebrafish or mitochondrial complex IV mutant, Surf^{-/-} zebrafish were pretreated with the drug compound ad Day 5, and co-treated with the drug compound and sodium azide, which is mitochondrial complex IV inhibitor at Day 6. Larval brain death, neuromuscular responses and heartbeat presence were tested at Day 7. Results are indicated for josamycin (FIG. 7(A)), cyproterone (FIG. 7(B)), felodipine (FIG. 7(C)), trapidil (FIG. 7(D)), and metyrapone (FIG. 7(E)), respectively. The data are plotted as the mean±standard deviation (n=3 biological replicates with 10 fish per treatment condition).

FIGS. 8(A) and 8(B) show protective effects of cyproterone (FIG. 8(A)) and felodipine (FIG. 8(B)) through swimming activity analysis. The data are plotted as the mean±standard deviation (n=8 individual fish). Statistical analyses were performed by Student's t-test (*p<0.05 vs. AB zebrafish).

FIGS. 9(A) and 9(B) show beneficial effects of cyproterone and trapidil through ATP content-based cell viability assay of primary mitochondrial diseases patients. Fibroblast cell lines of patients (Q1687F or Q1775F) with mitochondrial complex I or IV deficiency disorders and cells from healthy control (Q1881p1) were used. Results are indicated for trapidil (FIG. 9(A)) and cyproterone (FIG. 9(B)), respectively. Statistical analyses were performed by Student's t-test in Graphpad Prism (#p<0.05, and **p<0.01).

### DETAILED DESCRIPTION

In order to discover novel therapeutic uses of existing drugs for mitochondrial diseases, different AI-based in silico approaches were applied to 4 million existing compounds. A group of compounds were selected as drug compounds to treat or ameliorate mitochondrial dysfunction by using two different approaches based on transcriptional and structural similarities of three different seed groups. The attributes of the seed groups including abilities for the increase of mitochondrial biogenesis, the treatment of primary mitochondrial diseases, and the improvement of abnormalities in mitochondrial mechanisms, were found in the AI-drawn compounds. The protective abilities of selected compounds against mitochondrial defects or mitochondrial dysfunction were demonstrated empirically in human neuroblastoma cells. All of the compounds ameliorated mitochondrial membrane potential, oxidative stress, and ATP production as well as cell viability, and about a handful of compounds presented better recovery effect than phosphocreatine, the reference drug, in validation in vitro studies.

In the following description, numerous specific details are given to provide a thorough understanding of embodiments. The embodiments can be practiced without one or more of the specific details, or with other methods, components, materials, etc. In other instances, well-known structures, materials, or operations are not shown or described in detail to avoid obscuring aspects of the embodiments.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. The headings provided herein are for convenience only and do not interpret the scope or meaning of the embodiments.

The present description in some embodiments provides a method for enhancing mitochondrial function in a subject which comprises administering to such subject an effective amount of one or more compounds selected from the group consisting of:

josamycin, cyproterone, cilnidipine, felodipine, trapidil, metyrapone, and a pharmaceutically acceptable salt, an isomer, a hydrate, and a tautomer thereof.

Pharmaceutically acceptable salts are those salts which can be administered as drugs or pharmaceuticals to humans and/or animals and which, upon administration, retain at least some of the biological activity of the free compound (neutral compound or non-salt compound). The desired salt of the listed compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic peptides with amino acids, such as aspartate salts and glutamate salts, can also be prepared. The desired salt of an acidic peptide can be prepared by methods known to those of skill in the art by treating the compound with a base. Examples of inorganic salts of acidic peptides include, but are not limited to, alkali metal and alkaline earth salts, such as sodium salts, potassium salts, magnesium salts, and calcium salts; ammonium salts; and aluminum salts. Examples of organic salts of acidic peptides include, but are not limited to, procaine, dibenzylamine, N-ethylpiperidine, N,N′-dibenzylethylenediamine, and triethylamine salts. Salts of acidic peptides with amino acids, such as lysine salts, can also be prepared.

In some embodiments, a pharmaceutically acceptable salt of cyproterone is acetate. The present description in some embodiments provides a method for enhancing mitochondrial function in a subject, wherein enhancing mitochondrial function is enhancing cell viability. The present description in some embodiments provides a method for enhancing mitochondrial function in a subject, wherein enhancing mitochondrial function is increasing intracellular ATP content. The present description in some embodiments provides a method for enhancing mitochondrial function in a subject, wherein enhancing mitochondrial function is increasing mitochondrial membrane potential (ΔΨm). The present description in some embodiments provides a method for enhancing mitochondrial function in a subject, wherein enhancing mitochondrial function is reducing mitochondrial ROS. The present description in some embodiments provides a method for enhancing mitochondrial function in a subject, wherein enhancing mitochondrial function is reducing intracellular ROS. The present description in some embodiments provides a method for enhancing mitochondrial function in a subject, wherein enhancing mitochondrial function is reducing mitochondrial stress. The present description in some embodiments provides a method for enhancing mitochondrial function in a subject, wherein enhancing mitochondrial function is increasing lifespan. The present description in some embodiments provides a method for enhancing mitochondrial function in a subject, wherein enhancing mitochondrial function is enhancing neuronal activity. The present description in some embodiments provides a method for enhancing mitochondrial function in a subject, wherein enhancing mitochondrial function is enhancing locomotor activity. The present description in some embodiments provides a method for enhancing mitochondrial function in a subject, wherein enhancing mitochondrial function is enhancing growth.

The present description in some embodiments provides a method for enhancing mitochondrial function in a subject, wherein enhancing mitochondrial function is, but not limited to, one or more events selected from the group consisting of: (i) enhancing cell viability; (ii) increasing intracellular ATP content; (iii) increasing mitochondrial membrane potential (ΔΨm); (iv) reducing mitochondrial ROS; (v) reducing intracellular ROS; and (vi) reducing mitochondrial stress, compared to prior to administering the active compound(s).

The present description in some embodiments provides a method for enhancing mitochondrial function in a subject, wherein enhancing mitochondrial function is, but not limited to, one or more events selected from the group consisting of: (i) increasing lifespan, (ii) enhancing neuronal activity, (iii) enhancing locomotor activity; and (iv) enhancing growth, compared to prior to administering the active compound(s).

The present description in some embodiments provides a method for enhancing mitochondrial function in a subject, wherein the subject has one or more mitochondrial diseases. Particularly, the mitochondrial diseases comprise, but are not limited to, one or more selected from the group consisting of: Leber hereditary optic neuropathy (LHON), mitochondrial myopathy, encephalopathy, lactic acidosis and stroke-like episodes (MELAS) syndrome, mitochondrial complex I deficiency, mitochondrial complex II deficiency, mitochondrial complex III deficiency, mitochondrial complex IV deficiency, mitochondrial complex V deficiency, Leigh syndrome, autosomal dominant optic atrophy (ADOA), leukoencephalopathy with brain stem and spinal cord involvement and lactate elevation (LBSL), Luft disease, multiple acyl-CoA dehydrogenase (MAD) deficiency, mitochondrial enoyl CoA reductase protein-associated neurodegeneration (MEPAN) syndrome, mitochondrial DNA depletion, mitochondrial encephalopathy, pyruvate carboxylase deficiency, mitochondrial myopathy, Friedreich's ataxia, Barth syndrome, fatal infantile cardioencephalomyopathy, Charcot-Marie-Tooth disease, infantile lactic acidosis, congenital lactic acidosis (CLA), chronic lactic acidosis, Kearns-Sayre syndrome (KSS), mitochondrially inherited diabetes and deafness (MIDD), Alpers-Huttenlocher syndrome (AHS), childhood myocerebrohepatopathy spectrum (MCHS), ataxia neuropathy spectrum (ANS; previously referred to as mitochondrial recessive ataxia syndrome (MIRAS) and sensory ataxia neuropathy dysarthria and ophthalmoplegia (SANDO)), myoclonic epilepsy myopathy sensory ataxia (MEMSA; previously referred to as spinocerebellar ataxia with epilepsy (SCAE)), Sengers syndrome, MEGDEL syndrome (also known as 3-methylglutaconic aciduria with deafness, encephalopathy and Leigh-like syndrome), Pearson syndrome, myoclonic epilepsy with ragged red fibers (MERRF), neurogenic muscle weakness, ataxia and retinitis pigmentosa (NARP), chronic progressive external ophthalmoplegia (CPEO), mitochondrial neurogastrointestinal encephalopathy (MNGIE) syndrome, carnitine deficiency, carnitine-acylcarnitine translocase (CACT) deficiency, carnitine palm itoyl transferase 1A (CPT I) deficiency, carnitine palmitoyl transferase (CPT II) deficiency, creatine deficiency syndromes, creatine deficiency syndromes which contain guanidinoacetate methyltransferase (GAMT) deficiency, L-arginine:glycine amidinotransferase (AGAT) deficiency, or creatine transporter deficiency (including SLC6A8-related creatine transporter deficiency), thymidine kinase 2 deficiency (TK2D), pyruvate dehydrogenase complex deficiency (PDCD), fatty acid oxidation disorders (FAOD), fatty acid oxidation disorders which contain acyl-CoA dehydrogenase 9 (ACAD9) deficiency, multiple acyl-CoA dehydrogenase deficiency (MADD), long-chain acyl-CoA dehydrogenase (LCAD) deficiency, long-chain 3-hydroxyacyl-CoA dehydrogenase (LCHAD) deficiency, medium-chain acyl-CoA dehydrogenase (MCAD) deficiency, short-chain acyl-CoA dehydrogenase (SCAD) deficiency, short-chain 3-hydroxyacyl-CoA dehydrogenase (SCHAD) deficiency or very long-chain acyl-CoA dehydrogenase (VLCAD) deficiency, co-enzyme Q10 deficiency, and multiple mitochondrial dysfunction syndrome.

The present description in some embodiments provides a method for treating a mitochondrial disease by enhancing mitochondrial function in a subject, comprising administering to such subject an effective amount of one or more compounds selected from the group consisting of: josamycin, cyproterone, cilnidipine, felodipine, trapidil, metyrapone, and a pharmaceutically acceptable salt, an isomer, a hydrate, and a tautomer thereof. In some embodiments, a pharmaceutically acceptable salt of cyproterone is acetate. In some embodiments, the mitochondrial disease comprises, but is not limited to, one or more selected from the group consisting of: Leber hereditary optic neuropathy (LHON), mitochondrial myopathy, encephalopathy, lactic acidosis and stroke-like episodes (MELAS) syndrome, mitochondrial complex I deficiency, mitochondrial complex II deficiency, mitochondrial complex III deficiency, mitochondrial complex IV deficiency, mitochondrial complex V deficiency, Leigh syndrome, autosomal dominant optic atrophy (ADOA), leukoencephalopathy with brain stem and spinal cord involvement and lactate elevation (LBSL), Luft disease, multiple acyl-CoA dehydrogenase (MAD) deficiency, mitochondrial enoyl CoA reductase protein-associated neurodegeneration (MEPAN) syndrome, mitochondrial DNA depletion, mitochondrial encephalopathy, pyruvate carboxylase deficiency, mitochondrial myopathy, Friedreich's ataxia, Barth syndrome, fatal infantile cardioencephalomyopathy, Charcot-Marie-Tooth disease, infantile lactic acidosis, congenital lactic acidosis (CLA), chronic lactic acidosis, Kearns-Sayre syndrome (KSS), mitochondrially inherited diabetes and deafness (MIDD), Alpers-Huttenlocher syndrome (AHS), childhood myocerebrohepatopathy spectrum (MCHS), ataxia neuropathy spectrum (ANS; previously referred to as mitochondrial recessive ataxia syndrome (MIRAS) and sensory ataxia neuropathy dysarthria and ophthalmoplegia (SANDO)), myoclonic epilepsy myopathy sensory ataxia (MEMSA; previously referred to as spinocerebellar ataxia with epilepsy (SCAE)), Sengers syndrome, MEGDEL syndrome (also known as 3-methylglutaconic aciduria with deafness, encephalopathy and Leigh-like syndrome), Pearson syndrome, myoclonic epilepsy with ragged red fibers (MERRF), neurogenic muscle weakness, ataxia and retinitis pigmentosa (NARP), chronic progressive external ophthalmoplegia (CPEO), mitochondrial neurogastrointestinal encephalopathy (MNGIE) syndrome, carnitine deficiency, carnitine-acylcarnitine translocase (CACT) deficiency, carnitine palm itoyl transferase 1A (CPT I) deficiency, carnitine palmitoyl transferase (CPT II) deficiency, creatine deficiency syndromes, creatine deficiency syndromes which contain guanidinoacetate methyltransferase (GAMT) deficiency, L-arginine:glycine amidinotransferase (AGAT) deficiency, or creatine transporter deficiency (including SLC6A8-related creatine transporter deficiency), thymidine kinase 2 deficiency (TK2D), pyruvate dehydrogenase complex deficiency (PDCD), fatty acid oxidation disorders (FAOD), fatty acid oxidation disorders which contain acyl-CoA dehydrogenase 9 (ACAD9) deficiency, multiple acyl-CoA dehydrogenase deficiency (MADD), long-chain acyl-CoA dehydrogenase (LCAD) deficiency, long-chain 3-hydroxyacyl-CoA dehydrogenase (LCHAD) deficiency, medium-chain acyl-CoA dehydrogenase (MCAD) deficiency, short-chain acyl-CoA dehydrogenase (SCAD) deficiency, short-chain 3-hydroxyacyl-CoA dehydrogenase (SCHAD) deficiency or very long-chain acyl-CoA dehydrogenase (VLCAD) deficiency, co-enzyme Q10 deficiency, and multiple mitochondrial dysfunction syndrome.

### Definitions

The definitions of certain terms as used in this specification are provided below. Unless defined otherwise, all technical and scientific terms used herein generally have the same meaning as commonly understood by one of ordinary skill in the art to which this present technology belongs.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The term “biological sample” as used herein refers to a sample from the subject, and includes any bodily fluids, exudates, tissues or cells. Non-limiting examples include blood, plasma, serum, urine, tears, sputum, stool, saliva, nasal swabs, cells such as, but not limited to peripheral blood mononuclear cells (PBMCs), leukocytes, and tissue samples (e.g., biopsy samples). Samples can be fresh, frozen, or otherwise treated or preserved for evaluation by the methods disclosed herein.

As used herein the terms “enhancing” (or “enhance” or “enhancement”) is an approach for obtaining beneficial or desired results including clinical results. For example, enhancement can be of beneficial effect in a subject suffering from a disease or condition characterized by mitochondrial dysfunction, in that normalizing a biomarker of mitochondrial physiology may not achieve the optimum outcome for the subject; in such cases, enhancement of one or more biomarkers of mitochondrial physiology can be beneficial, for example, higher-than-normal levels of ATP, or lower-than normal levels of lactic acid (lactate) can be beneficial to such a subject.

As used herein, the terms “treating” (or “treat” or “treatment”) is an approach for obtaining beneficial or desired results including clinical results. For purposes of the present description, beneficial or desired clinical results include, but are not limited to, alleviating one or more symptoms of a disease, such as a mitochondrial disease, reducing one or more symptoms of a disease, such as a mitochondrial disease, preventing one or more symptoms of a disease, such as a mitochondrial disease, treating one or more symptoms of a disease, such as a mitochondrial disease, ameliorating one or more symptoms of a disease, such as a mitochondrial disease, delaying the onset of one or more symptoms of a disease, such as the onset of a mitochondrial disease, diminishing the extent of one or more symptoms of a disease, such as the extent of a mitochondrial disease, stabilizing a disease, such as a mitochondrial disease, delaying or slowing the progression of a disease, such as the progression of a mitochondrial disease, increasing the quality of life of the subjects suffering from a disease, such as the quality of life of the subject suffering from a mitochondrial disease, and/or prolonging survival of the subjects. In some embodiments, for example, a subject is successfully “treated” for a disease or disorder characterized by mitochondrial dysfunction if, after receiving a therapeutic amount of the active agents according to the methods described herein, the subject shows observable and/or measurable reduction in the disruption of mitochondrial oxidative phosphorylation. It is also to be appreciated that the various modes of treatment or prevention of medical diseases and conditions as described are intended to mean “substantial,” which includes total but also less than total treatment or prevention, and wherein some biologically or medically relevant result is achieved.

As used herein, the terms “effective amount” or “therapeutically effective amount” refer to an amount of a compound, composition or drug sufficient to enhance a specified function, such as enhance or increase desired and/or beneficial function, reduce or decrease undesired function; or to treat a specified disease, condition, or disease, such as ameliorate, palliate, lessen, and/or delay one or more of its symptoms. For example, in reference to a mitochondrial disease, an effective amount comprises an amount sufficient to delay development of a mitochondrial disease or some of its potential multi-system clinical features. In some embodiments, the effective amount is an amount sufficient to prevent or delay recurrence or progression. An effective amount can be administered in one or more administrations. For example, in the case of mitochondrial diseases, the effective amount of the compound, composition or drug may: (i) inhibit, retard, slow to some extent and preferably stop muscular dysfunction; (ii) inhibit, retard, slow to some extent and preferably stop neurological dysfunction; (iii) inhibit, retard, slow to some extent respiratory dysfunction; (iv) inhibit, retard, slow to some extent morbidity; (v) prevent or delay occurrence and/or recurrence of a mitochondrial disease; and/or (vi) relieve to some extent one or more of the symptoms associated with having a mitochondrial disease.

As used herein, the “administration” of an agent, drug, or compound to a subject includes any route of introducing or delivering to a subject the agent, drug, or compound to perform its intended function. For the purposes of the present description, the administering is conducted, but not limited to, intravenously, intraportally, intra-arterially, intraperitoneally, intrahepatically, by hepatic arterial infusion, intravesicularly, subcutaneously, intrathecally, intrapulmonarily, intramuscularly, intratracheally, intraocularly, transdermally, intradermally, topically, orally, or by inhalation. Administration includes self-administration and the administration by another. As used herein, the term “subject” (or “subjects”) may be a human or non-human subject, such as, not limited to, a nonhuman primate, a dog, a cat, a horse, a rodent, a rabbit, etc.

It is understood that aspects and embodiments of the present description described herein include “consisting” and/or “consisting essentially of” aspects and embodiments. As used herein and in the appended claims, the singular forms “a,” “or,” and “the” include plural referents unless the context clearly dictates otherwise.

Active Ingredient Compounds and Compositions

In embodiments, one or more selected from the group consisting of josamycin, cyproterone, cilnidipine, felodipine, trapidil, metyrapone, and a pharmaceutically acceptable salt, an isomer, a hydrate, and a tautomer thereof are employed as an active ingredient. Pharmaceutically acceptable salts are those salts which can be administered as drugs or pharmaceuticals to humans and/or animals and which, upon administration, retain at least some of the biological activity of the free compound (neutral compound or non-salt compound). The desired salt of the listed compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic peptides with amino acids, such as aspartate salts and glutamate salts, can also be prepared. The desired salt of an acidic peptide can be prepared by methods known to those of skill in the art by treating the compound with a base. Examples of inorganic salts of acidic peptides include, but are not limited to, alkali metal and alkaline earth salts, such as sodium salts, potassium salts, magnesium salts, zinc salts, copper salts, ferric salts, and calcium salts; ammonium salts; and aluminum salts. Examples of organic salts of acidic peptides include, but are not limited to, procaine, dibenzylamine, N-ethylpiperidine, N,N′-dibenzylethylenediamine, and triethylamine salts. Salts of acidic peptides with amino acids, such as lysine salts, can also be prepared.

In some embodiments, a pharmaceutically acceptable salt of cyproterone is acetate. When two or more active ingredients are employed, the active ingredients may be separately, sequentially, or simultaneously. As used herein, the term “separately” refers to an administration of at least two active ingredients at the same time or at substantially the same time by different routes. As used herein, the term “sequentially” refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. As used herein, the term “simultaneously” refers to the administration of at least two active ingredients by the same route and at the same time or at substantially the same time.

The active agents described herein can be incorporated into pharmaceutical compositions for administration, singly or in combination, to a subject for the treatment or prevention of a medical disease or condition described herein, such as mitochondrial dysfunction associated conditions and symptoms. Such compositions typically include the active agent (e.g., josamycin, cyproterone, cilnidipine, felodipine, trapidil, metyrapone, and a pharmaceutically acceptable salt, an isomer, a hydrate, and a tautomer thereof) and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., treatment for one week).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor, or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi. The composition can include a carrier, which can be a solvent or dispersion medium containing, for example, water, ethanol, polyol (for example, glycerol, propylene glycol, liquid polyethylene glycol, and the like), and suitable mixtures thereof.

The composition may include various antibacterial and antifungal agents, for example, parabens, chlorobutanol, phenol, ascorbic acid, thiomerasol, and the like. Glutathione and other antioxidants can be included to prevent oxidation. The composition also may include isotonic agents such as, for example, sugars, polyalcohols such as mannitol, sorbitol, or sodium chloride in the composition. Injectable compositions may include an agent that delays absorption, for example, aluminum monostearate or gelatin, to prolong absorption.

The composition may be in a sterile injectable solution that can be prepared by incorporating the active ingredient compound in the required amount in an appropriate solvent with one or a combination of ingredients enumerated above, as required, followed by filtered sterilization. For example, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, primogel, or corn starch; a lubricant such as magnesium stearate or sterotes; a glidant such as colloidal silicon dioxide; a sweetening agent such as sucrose, trehalose, or saccharin; or a flavoring agent such as peppermint, methyl salicylate, or citrus (orange) or other fruit (grapefruit, grape, apple, pineapple, and the like) flavoring agent.

For administration by inhalation, the active agent such as an aromatic-cationic peptide of the present technology can be delivered in the form of an aerosol spray from a pressurized container or dispenser, which contains a suitable propellant, e.g., a gas such as carbon dioxide, or a nebulizer.

Systemic administration of an active agent such as an aromatic-cationic peptide of the present technology as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds arc formulated into ointments, salves, gels, or creams as generally known in the art. In one embodiment, transdermal administration may be performed by iontophoresis.

The active compound can be formulated in a carrier system. The carrier can be a colloidal system. The colloidal system can be a liposome, a phospholipid bilayer vehicle.

In one embodiment, the therapeutic agent such as an aromatic-cationic peptide is encapsulated in a liposome while maintaining peptide integrity. One skilled in the art would appreciate that there are a variety of methods to prepare liposomes. Liposomal formulations can delay clearance and increase cellular uptake. See e.g., Reddy, Ann. Pharmacother., 34(7-8):915-923 (2000)). The active compound can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

Examples of polymer microsphere sustained release formulations are described in, for example, PCT publication WO 99/15154 (Tracy, et al.), U.S. Pat. Nos. 5,674,534 and 5,716,644 (both to Zale, el al.).

In some embodiments, the active compounds may be prepared with carriers that will protect the active compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques.

Typically, an effective amount of the active compound, sufficient for achieving a therapeutic or prophylactic effect, ranges from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. In one embodiment, a single dosage of an active compound ranges from 0.001-10,000 micrograms per kg body weight.

These and other aspects and advantages of the present invention will become apparent from the subsequent detailed description and the appended claims. It is to be understood that one, or all of the properties of the various embodiments described herein may be combined to form other embodiments of the present description.

### EXAMPLE

The presently disclosed subject matter will be better understood by reference to the following Example, which is provided as exemplary of the presently disclosed subject matter, and not by way of limitation.

### Example 1: AI-Aided Discovery of Repurposed Drug Compounds for Mitochondrial Diseases

Method

In order to screen compounds that are suitable for enhancing mitochondrial function and/or treating mitochondrial disease(s), a set of AI-based in silico approaches were applied to 4 million existing compounds. In silico-based approaches to screen active compounds for mitochondrial diseases, which were applied to the known compounds, are below.

Module 1—Graphic knowledge database

Module 2—Drug repurposing model based on the drug-perturbed transcriptional expression

pattern of genes

Module 3—Clustering model of the pathophysiological property-embodied latent space

of Module 2

Module 4—Drug repurposing model based on structural and drug-like similarity

Module 5—Drug-focused information searching model

The 1^{st} Seed Group

In order to be applied to AI-based drug repurposing models as seed compounds, three therapeutic compounds, bezafibrate, curcumin, and resveratrol, which are known to increase mitochondrial biogenesis were selected from literatures (Hirano M et al., Emerging therapies for mitochondrial diseases. Essays Biochem. 2018; 62(3):467-481; El-Hattab A W et al., Therapies for mitochondrial diseases and current clinical trials. Mol Genet Metab. 2017; 122(3):1-9; Nightingale H. et al., Emerging therapies for mitochondrial diseases. Brain. 2016; 139(6):1633-1648).

Further, additional four drugs, acipimox, pioglitazone, rosiglitazone, and varenicline, were selected by Module I, and added to the 1^{st} seed group in order to enhance the property for mitochondrial biogenesis in the 1^{st} seed group. The four drugs that Module 1 suggested have similar connecting properties with the above three drugs (bezafibrate, curcumin, and resveratrol) in terms of drug-targets, drug-pathways, and disease-pathways.

The association of these compounds to the mitochondrial pathophysiology was proved by literature; acipimox showed a beneficial effect on muscle mitochondrial function as an NAD⁺ precursor in clinical study (van de Weijer T et al., Evidence for a direct effect of the NAD⁺ precursor acipimox on muscle mitochondrial function in humans. Diabetes. 2015; 64(4):1193-1201) and entered a new clinical study (NCT03325491) for muscle performance with impaired mitochondrial function after the positive result of previous clinical study with muscle performance in aged men (NCT02792621). Pioglitazone and rosiglitazone are reported to induce mitochondrial biogenesis as a peroxisome proliferator-activated receptor (PPAR) gamma agonist in adipose tissue and patient cells with Friedreich's ataxia and Down syndrome (Bogacka I. et al., Pioglitazone Induces Mitochondrial Biogenesis in Human Subcutaneous Adipose Tissue In Vivo. Diabetes. 2005; 54(5):1392-1399; Aranca T V. et al., Emerging therapies in Friedreich's ataxia. Neurodegener Dis Manag. 2016; 6(1):49-65; Mollo N. et al., Pioglitazone Improves Mitochondrial Organization and Bioenergetics in Down Syndrome Cells. Front Genet. 2019; 10). Varenicline is a partial agonist of the alpha 4/beta 2 subtype of the nicotinic acetylcholine receptor. Clinical trials of varenicline (NCT00803868) and pioglitazone (NCT00811681) have conducted for Friedreich's ataxia, a neurodegenerative movement disease with a genetic defect in FXN whose encoded protein has an essential role for mitochondrial respiratory complexes. Therefore, these four therapeutic compounds (acipimox, pioglitazone, rosiglitazone, and varenicline) were designated to a mitochondrial biogenesis seed group together with three known therapeutic compounds mentioned above (bezafibrate, curcumin, and resveratrol).

The 2^{nd} Seed Group and the 3^{rd} Seed Group

The therapeutic molecules found in reported clinical trials for mitochondrial diseases were collected, and 13 molecules were selected as the second seed group (TABLE 1).

TABLE 1

| Seed | Mechanism | Reference |
|---|---|---|
| CoQ10 | OXPHOS/ROS | NCT00432744 |
| EPI-743 | Antioxidant | NCT01370447 |
| Idebenone | Antioxidant | NCT00887562 |
| L-Arginine | NO precursor | NCT01603446 |
| Lipoic acid | Antioxidant | NCT02348125 |
| Bezafibrate | Mitochondrial biogenesis | NCT02398201 |
| Curcumin | Antioxidant, | NCT00528151 |
| | Mitochondrial biogenesis | |
| Cyclosporine A | Inhibition of | NCT02176733; |
| | mitochondrial PTP | NCT02616484 |
| RP103 | Cystine-depleting agent | NCT02023866; |
| | | NCT02473445 |
| Omaveloxolone | Antioxidant, | NCT0225542 |
| | Nrf2 Activator, | |
| | NFkb Inhibitor | |
| KH176 | Antioxidant | NCT02909400; |
| | | NCT02544217 |
| KL1333 | NAD+ modulator | NCT03056209 |
| Pyruvate | NAD donor | JMA-IIA00093 |

This seed group of the reported clinical trial drugs consisted of antioxidants, mitochondrial biogenesis boosters, NAD⁺ modulators, and so on. Antioxidant and oxidative stress-modulating compounds were the most dominant.

To supplement recent pathological, physiological, and pharmaceutical advances in mitochondrial biology, the third seed group was prepared additionally. Drug compounds in reported preclinical and clinical trials for neurodegenerative diseases, oxidative stress-induced diseases, or metabolic diseases were applied to Module 1 to identify compounds with the mitochondrial mechanism of action (MOA). The diseases were used for Module 1 are including but not limited to Parkinson's disease, Alzheimer's disease, amyotrophic lateral sclerosis, multiple sclerosis, ischemia-reperfusion injury, NASH, cardiovascular diseases, chronic obstructive pulmonary disease, chronic kidney disease, and diabetes. In addition, the therapeutic molecules designed for emerging targets for mitochondrial diseases were interrogated to Module 1. The emerging targets noted for therapy of mitochondrial diseases included, but were not limited to, DRP1 inhibitors, 5-lipoxygenase inhibitors, SIRT1 activators, PARP-1 inhibitors, and NRF2 activators. As a result, among therapeutic molecules suggested by Module 1, ten compounds whose mitochondrial MOAs were proved in in vivo models were chosen as a mitochondrial MOA seed group (the 3^{rd} seed group). They are imeglimin (Vial G. et al., Imeglimin Normalizes Glucose Tolerance and Insulin Sensitivity and Improves Mitochondrial Function in Liver of a High-Fat, High-Sucrose Diet Mice Model. Diabetes. 2015; 64(6):2254-2264), blarcamesine (Missling CU, ANAVEX 2-73, a Clinical Candidate for Neurodevelopmental Disorders: New data Including AnC-Seizure data in Angelman Syndrome. 24), isradipine (Guzman J N, et al., Systemic isradipine treatment diminishes calcium-dependent mitochondrial oxidant stress. J Clin Invest. 2018; 128(6):2266-2280), latrepirdine (Eckert S H, et al., Mitochondrial Pharmacology of Dimebon (Latrepirdine) Calls for a New Look at its Possible Therapeutic Potential in Alzheimer's Disease. Aging Dis. 2018; 9(4):729-744), resveratrol (Decui L, et al., Micronized resveratrol shows promising effects in a seizure model in zebrafish and signalizes an important advance in epilepsy treatment. Epilepsy Res. 2020; 159:106243), nicotinamide (Bayrakdar E T, et al., Ex vivo protective effects of nicotinamide and 3-aminobenzamide on rat synaptosomes treated with Aβ(1-42). Cell Biochem Funct. 2014; 32(7):557-564), Mdivi-1 (Bido S, et al., Mitochondrial division inhibitor-1 is neuroprotective in the A53T-α-synuclein rat model of Parkinson's disease. Sci Rep. 2017; 7), CNB-001 (Jayaraj R L, et al., CNB-001 a novel curcumin derivative, guards dopamine neurons in MPTP model of Parkinson's disease. Biomed Res Int. 2014; 2014:236182), CPUY192018 (Lu M-C, et al., CPUY192018, a potent inhibitor of the Keap1-Nrf2 protein-protein interaction, alleviates renal inflammation in mice by restricting oxidative stress and NF-κB activation. Redox Biol. 2019; 26; Lu M C, et al., An inhibitor of the Keap1-Nrf2 protein-protein interaction protects NCM460 colonic cells and alleviates experimental colitis. Sci Rep. 2016; 6), and compound 14 (Ma B, et al., Design, synthesis and identification of novel, orally bioavailable non-covalent Nrf2 activators. Bioorganic & Medicinal Chemistry Letters. Published online Dec. 2, 2019:126852).

Assessment of Candidate Drug Compounds for Mitochondrial Diseases Treatment Efficacy by Using Drug-Perturbed Transcriptional Similarity

The mitochondrial biogenesis seed group (the 1^{st} seed group) was applied to Module 2 and Module 3. Module 2 is a drug repurposing model based on transcriptional similarity by training drug-perturbed gene expression datasets obtained from Broad Institute LINCS project (Subramanian A., et al., A Next Generation Connectivity Map: L1000 Platform And The First 1,000,000 Profiles. bioRxiv. Published online May 10, 2017:136168; Musa A., et al., L1000 Viewer: A Search Engine and Web Interface for the LINCS Data Repository. Front Genet. 2019; 10). It predicts new uses of more than 11,000 drugs in the LINCS library. Module 3 was created because the mitochondrial diseases were not classified in the drug indication system of Module 2. In Module 3, each given vector of the drug perturbed gene expression data in the latent space of Module 2 under optimized hyper-parameters was trained and clustered. The distribution pattern and the frequency of the gene expression data of a drug in the clusters were reflected in our scoring system. As a consequence, so-called neighbor compounds were drawn from Module 3, by using drug-perturbed transcriptional similarity of a seed compound. Indeed, a drug-target similarity was validated in silico between seed compounds and neighbor compounds when anticancer drugs in a cancer validation set were applied to Module 3.

Assessment of Candidate Drug Compounds for Mitochondrial Diseases Treatment Efficacy by Using Structural and Drug-Like Similarity

Module 4 was constructed by using a dataset of about 4 million SMILES strings with physical activities from public databases. The deep learning algorithm of Module 4 created a latent chemical space in which structural and drug-like similarity of compounds is embodied, and it is allowed discovering neighbor compounds with desired properties from a seed compound.

The clinical trial seed group (the 2^{nd} seed group) and the mitochondrial MOA seed group (the 3^{rd} seed group) were applied to Module 4. The drug compounds from the clinical trial seed group were selected by calculation of cosine distance and the drug compounds list from the mitochondrial MOA seed group was drawn by computing both cosine distance and Euclidean distance. The neighbor compounds of each seed were identified by mapping their SMILES with CAS registry numbers. The compounds with no CAS registry number were eliminated from the lists.

Filtering

All the neighbor compounds were applied to Module 5, which enabled researchers to search drug-focused information in scientific literature and patents. Module 5 removed the compounds that were reported as therapeutics on the primary mitochondrial diseases attributed to dysfunctions in mitochondrial respiratory chain reaction, demonstrated to have mitochondrial toxicity by empirical tests, and reported to be toxic to patients with mitochondrial diseases or to mitochondrial function and/or mitochondrial homeostasis including, but not limited to the cellular events such as apoptosis, ROS production, and inhibition of mitochondrial respiratory complexes. Additionally, compounds with a toxicity warning or developmental disadvantages including preclinical, parenteral, commercially not available, and having severe side effect were filtered out.

Results

A list of neighbor compounds was drawn through STANDIGM AI-based drug repurposing process by using 27 seed compounds for their therapeutic and/or mechanistic activities for mitochondrial diseases or mitochondria-related diseases.

After the filtering process, three drug compounds (i.e., josamycin, cyproterone, and cilnidipine) were selected through the approach of using structural similarity with the clinical trial seed group, and three drug compounds (i.e., felodipine, trapidil, and metyrapone) were selected through the approach of using structural similarity with the mitochondrial MOA seed group.

Table 2 shows six (6) compounds selected from each approach with its IUPAC name and chemical structure. In Example 2 below, cyproterone was tested as a salt form, and its salt form is also described in Table 2. However, the pharmaceutically acceptable salts of six (6) compounds are not limited to the salt forms described in Table 2.

TABLE 2

| | | | Com- | |
|---|---|---|---|---|
| | | | pound | |
| | | | used in | |
| | | | experi- | |
| Name | IUPAC name | Structure | ments | IUPAC name |
| josamy- cin | [(2S,3S,4R,6S)-6- [(2R,3S,4R,5R,6S)-6- [[(4R,5S,6S,7R,9R,10R,11E,13E,16R)- 4-acetyloxy-10-hydroxy-5-methoxy- 9,16-dimethyl-2-oxo-7-(2-oxoethyl)- 1-oxacyclohexadeca-11,13-dien-6- yl]oxy]-4-(dimethylamino)-5-hydroxy- 2-methyloxan-3-yl]oxy-4-hydroxy-2,4- dimethyloxan-3-yl]3-methylbutanoate | 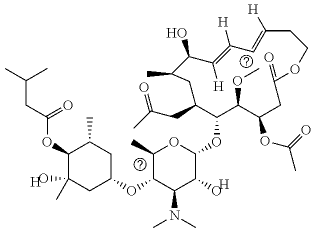 | josa- mycin | |
| cyproter- one | (1S,2S,3S,5R,11R,12S,15R,16S)- 15-acetyl-9-chloro-15-hydroxy- 2,16-dimethylpentacyclo [9.7.0.02,8.03,5.012,16]octadeca- 7,9-dien-6-one | 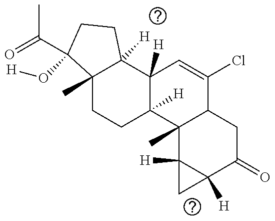 | cyproter- one ace- tate | [(1S,2S,3S,5R,11R,12S,15R,16S)- 15-acetyl-9-chloro-2,16-dimethyl- 6-oxo-15-pentacyclo [9.7.0.02,8.03,5.012,16]octadeca- 7,9-dienyl]acetate |
| cilni- dipine | 3-O-(2-methoxyethyl)5-O[(E)-3-phen- ylprop-2-enyl]2,6-dimethyl-4-(3- nitrophenyl)-1,4-dihydropyridine-3,5- dicarboxylate | 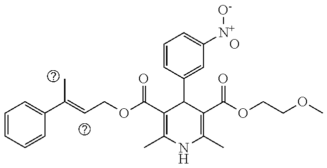 | cilini- dipine | |
| felodi- pine | 5-O-ethyl 3-O-methyl 4-(2,3-dichloro- phenyl)-2,6-dimethyl-1,4- dihydropyridin-3,5-dicarboxylate | 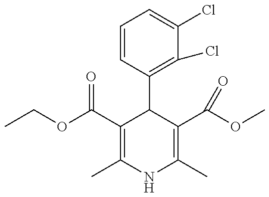 [SMILES: CCOC(=O)C1=C(C)NC(C)=C(C(=O)OC)C1c1cccc(Cl)c1Cl] | felodi- pine | |
| trapidil | N,N-diethyl-5-methyl- [1,2,4]triazolo[1,5- a]pyrimidin-7-amine | 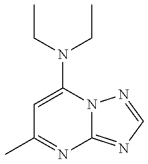 | trapidil | |
| metyra- pone | 2-methyl-1,2-dipyridin-3- ylpropan-1-one | 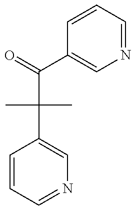 [SMILES: CC(C)(C(=O)c1cccnc1)c1cccnc1] | metyra- pone | |

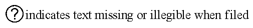

### Example 2: In Vitro Validation of AI-Discovered Drug Compounds for Mitochondrial Diseases Treatment Efficacy

Method

Cell Culture and Chemical Treatment

SH-SY5Y human neuroblastoma cells were cultured in Dulbecco's Modified Eagle Medium (DMEM)/F12 supplemented with 10% fetal bovine serum (FBS), 100 U/ml penicillin, and 100 μg/ml streptomycin (complete media, CM) at 37° C./5% CO₂. Cells seeded at 5×10⁴ cells/well were cultured in 96-well plates for 24 hours followed by incubation in serum-deficient media (SDM, DMEM/F12 containing 0.5% FBS) for 16 hours. Cells in SDM were pre-treated with each chemical for 4 hours, followed by incubation with 1 mM mitochondrial complex I inhibitor (MPP⁺) or dimethyl sulfoxide (DMSO) vehicle for hours.

Cell Viability Assay—Methylthiazoletetrazolium (MTT) Assay

MTT assay is a measure of a mitochondrial NAD(P)H-dependent oxidoreductase activity to reduce MTT to formazan in live cells. Thus, the MTT assay examines viable cell amount by measuring the mitochondrial metabolic rate (Rai Y. et al., Mitochondrial biogenesis and metabolic hyperactivation limits the application of MTT assay in the estimation of radiation induced growth inhibition, Scientific Reports. 2018; 8(1):1531), presenting the metabolic viability of cells. SH-SY5Y cells (2.5×10⁴ cells/well) in SDM in 96-well plates were treated with chemicals as indicated by the previous paragraph and further incubated with 3-(4,5 dimethylthiazol-2-yl)-2,5-diphenyl-tetrazolium bromide (MTT, Sigma) solution (0.2 mg/ml MTT in PBS) for 4 hours. The MTT formazan precipitates formed by live cells were dissolved in 100 μl of 0.04 N HCl/isopropanol. The absorbance at 540 nm was measured by ELISA microplate reader (Molecular Devices, Sunnyvale, CA).

Intracellular ATP Assay—Luciferase-Based Assay

Intracellular ATP content was measured by luciferin-luciferase reaction with Cell Titer luciferase kit (Promega) according to the manufacturer's instructions. 20 μl cell lysates were mixed with 20 μl of the luciferin-luciferase reaction buffer and incubated at 20° C. for 10 min. The luminescence signal was measured with an LB 9501 LUMAT® luminometer (Berthold, German). It was subtracted (i.e., corrected) from the signal that a background luminescence value in control wells containing medium without cells. The amounts of ATP content were normalized to protein concentration. All data were presented as a percent of control after calculation.

Mitochondrial Membrane Potential (ΔΨm) Assay and Reactive Oxygen Species (ROS) Assay

Mitochondrial membrane potential was measured using tetramethylrhodamine Ethylester (TMRE, Molecular Probe). The cells in black 96-well culture plate were incubated with 200 nM TMRE and HOECHST® 33342 (0.5 μM) for 30 min at 37° C. in phenol red-free SDM.

Similarly, total and mitochondrial ROS generations were measured using CM-H2DCFDA and MitoSox, respectively. Cells were incubated with 1 μM CM-H2DCFDA or 5 μM MitoSox and 0.5 μM Hoechst 33342 for 1 hour at 37° C.

Fluorescence intensities at 550 nm/580 nm for TMRE, at 510 nm/580 nm for MitoSox, or at 494 nm/522 nm for CM-H2DCFDA were normalized by HOECHST® intensity at 355 nm/480 nm (Spectramax Gemini EM, Molecular Devices, Sunnyvale, CA, USA).

Results

Three (3) Drug Compounds (Josamycin, Cyproterone, and Cilnidipine) Showed Protective Effects

Quiescent human neuroblastoma cells (SH-SY5Y) cells were treated with each drug compounds at various concentrations (i.e., 1 nM, 1 μM) for 4 hours before the treatment of 1 mM MPP⁺ for 20 hours.

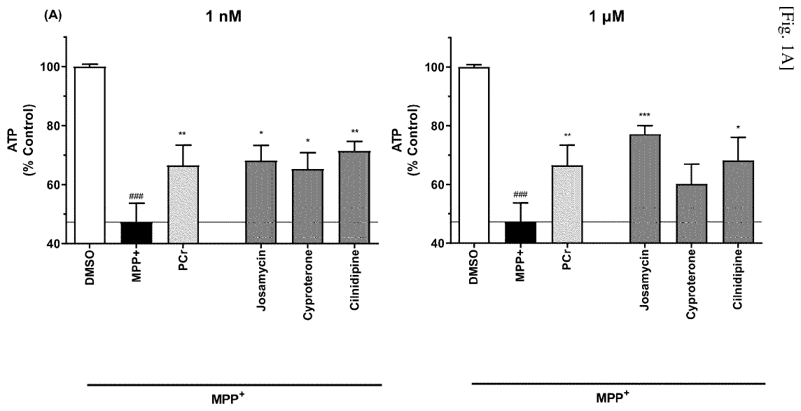

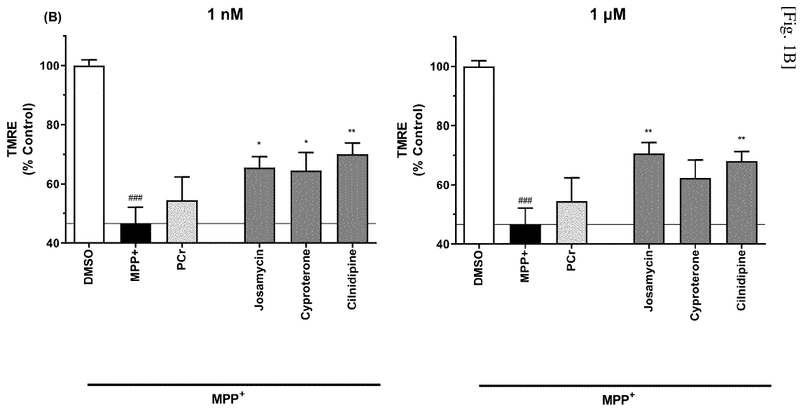

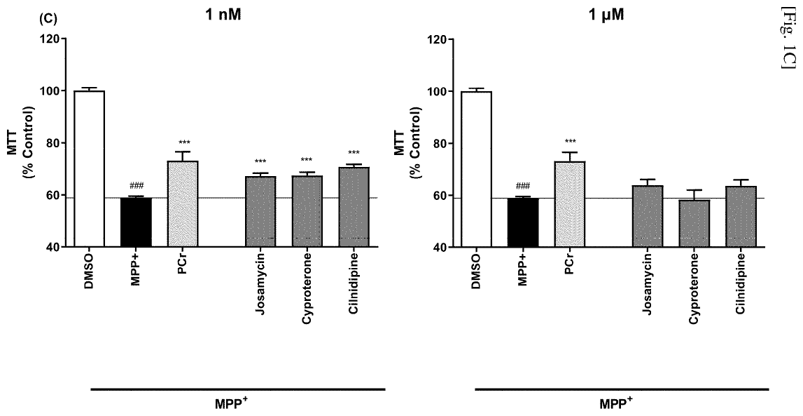

As shown in FIGS. 1(A)-1(C), the MPP⁺ induced cellular and mitochondrial damages in human neuroblastoma cells (SH-SY5Y) were alleviated by all three compounds, josamycin, cyproterone acetate, and cilnidipine. Specifically, as shown in FIGS. 1(A) and 1(B), all three compounds showed their protective effects against MPP⁺ in intracellular ATP content and TMRE-mediated mitochondrial membrane potential. Especially, most of the compounds presented similar or even better protection ability than phosphocreatine, a reference and therapeutic compound for mitochondrial diseases in clinical trials.

As shown in FIG. 1(C), all three drugs increased the metabolic cell viability against the MPP⁺-induced death. Specifically, the beneficial effect of all three compounds was statistically significant at 1 nM.

Five (5) Drug Compounds (Josamycin, Cilnidipine, Felodipine, Trapidil, and Metyrapone) Showed Protective Effects in a Dose-Dependent Manner

Five drug compounds, josamycin, cilnidipine, felodipine, trapidil, and metyrapone were tested to validate their protective effect in a dose-dependent manner (i.e., 0.001 to 10 μM for josamycin, and cilnidipine, and 0.01 to 100 nM for felodipine, trapidil, and metyrapone).

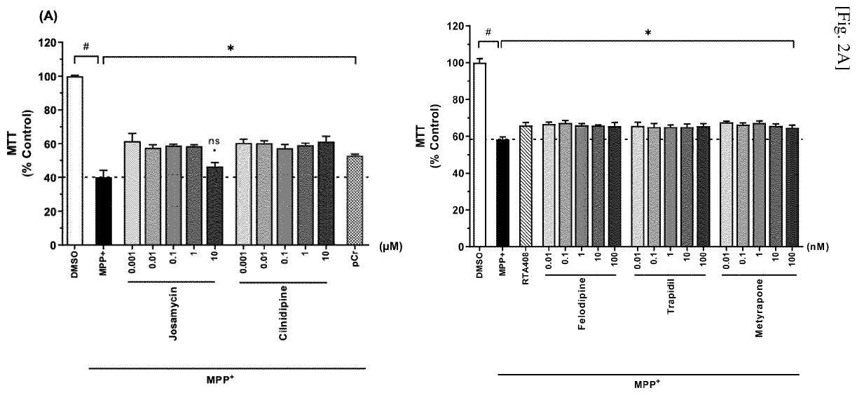

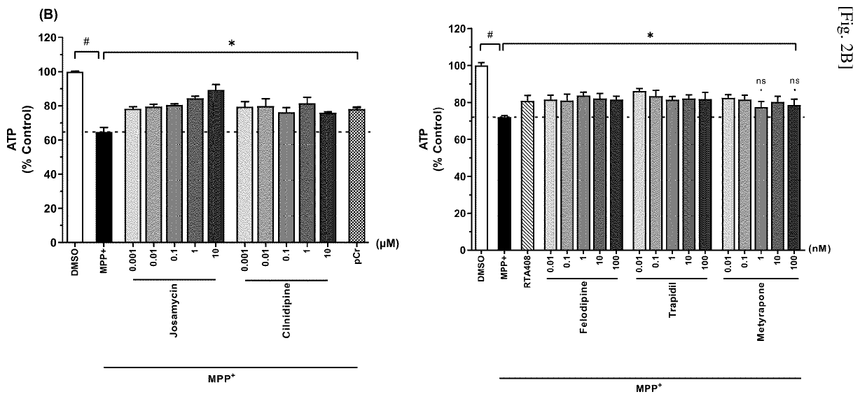

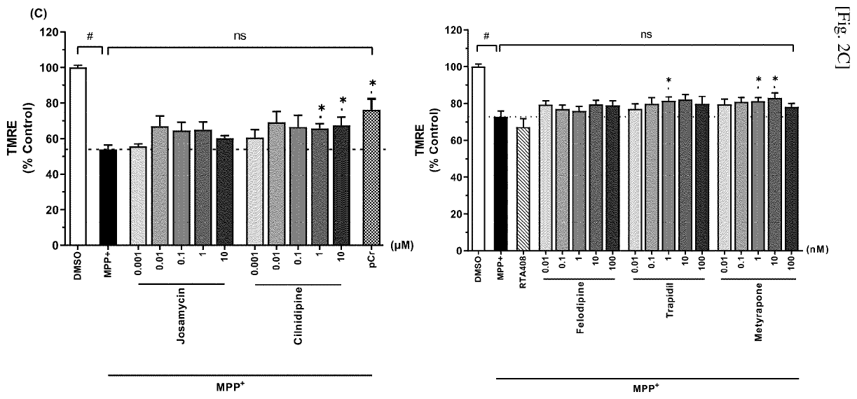

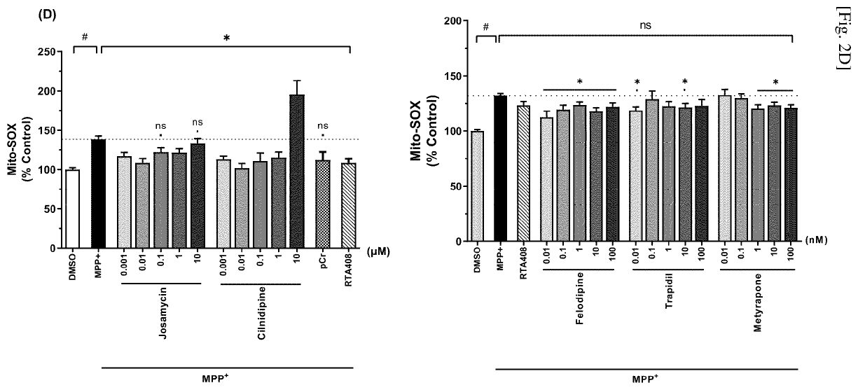

As shown in FIGS. 2(A) and 2(B), all of the drug compounds showed their protective effects against MPP⁺ in cell viability, and intracellular ATP content, respectively throughout all the tested range of the concentrations. Interestingly, josamycin and cilnidipine presented better recovering ability than phosphocreatine, a reference drug for mitochondrial diseases in clinical trials. Felodipine, trapidil, and metyrapone showed the similar effects to RTA408, another reference drug for mitochondrial diseases in clinical trial. As shown in FIG. 2(C), the reduced TMRE-mediated mitochondrial membrane potential by MPP⁺ was alleviated when the five drug compounds were treated throughout all the tested range of concentrations.

As shown in FIGS. 2(D) and 2(E), all five drug compounds, josamycin, cilnidipine, felodipine, trapidil, and metyrapone showed reduced ROS production compared to MPP⁺-lesioned cells throughout the tested range of concentrations. The levels of reduction were similar to or better than the levels of two reference trial drugs, phosphocreatine and RTA408. Interestingly, cilnidipine presented significant reduction of both mitochondrial and cellular ROS production in 0.001 to 1 μM concentrations but the ROS production increased dramatically in 10 μM.

### Example 3: In Vivo Validation with Mitochondrial Disease Animal Models, Namely C. elegans (Worms) and D. rerio (Zebrafish), of AI-Discovered Drug Compounds for Treating Mitochondrial Diseases

Method

Lifespan Assay in C. elegans—Activity, Mortality, and Body Length

Lifespan assay was performed on mitochondrial complex I mutant, C. elegans gas-1 (fc21) and C. elegans wild-type (Bristol N2). The Bristol N2 and gas-1(fc21) populations were synchronized by bleaching and grown to L4 larval stage on nematode growth media (NGM) plates. 30-35 L4 hermaphrodites were sorted into each well, in a 24-well plate by the COPAS BIOSORTER™. The 24-well plates consisted of NGM, E. coli OP50 as food for C. elegans, 50 μM of fluorodeoxyuridine (FUDR) to prevent progeny development of C. elegans, and the appropriate concentration of the drug compounds. The 24-well plates were then imaged daily by the EPSON PERFECTION™ V700 Flatbed Scanner with two consecutive images throughout the animals' lifespan. The light from the scanner caused physical stimulation in C. elegans to determine whether C. elegans were alive. A difference image was calculated from each pair of image sets, which reflected the area of C. elegans movement in each well, and the obtained image was then converted to a binary, black and white, image through Hysteresis thresholding. The WormScan score, which was generated based on pixel difference, measured an integrated metric of lifespan and activity value. C. elegans were scored as dead if their movement was less than 10%. The body length or mortality of C. elegans were conducted on Image J. Statistical analyses were performed in Graphpad Prism V8.

Mitochondrial Stress Assay—ARM-6 and HSP-6 Fluorescence

Hsp-6::GFP induction was quantified in living C. elegans arm-6; hsp-6; gas-1(fc32) (AGH) animals using the Cell Insight CX5 High Content Screening (HCS) Platform (Thermo Scientific) to evaluate mitochondria stress. To quantify hsp-6::GFP induction, the C. elegans AGH population was synchronized and grown from birth to L1 stage on solid NGM plates. C. elegans were then transferred to 384-well plates and grown in 50 μL of E. coli OP50 media with 25 μM of the drug compound. Subsequently, C. elegans were grown in the 384-well plates from L1 to L4 larval stages. At the L4 stage, C. elegans were paralyzed with 40 μL of 2.4 mg/mL levamisole. High content imaging analysis was used to image the living C. elegans AGH. Arm-6 was used as a body size marker, and its blue fluorescence induction was quantified at 386 nm excitation wavelength. Hsp-6::GFP induction was a green fluorescence marker of mitochondrial stress and was quantified at 485 nm excitation wavelength. The fluorescent intensity from Hsp-6::GFP induction was normalized to body size of the C. elegans AGH worm strain, to analyze the effectiveness of drug treatment(s). Statistical analyses were performed in Graphpad Prism V8.

TMRE Assay—Mitochondrial Membrane Potential

High-throughput quantification of mitochondrial membrane potential and mass in C. elegans was quantified in C. elegans Cox-4::GFP (green fluorescence protein) worms using the COPAS Biosorter. Mitochondrial membrane potential was measured with the red fluorescent dye, TMRE. C. elegans Cox-4::GFP were synchronized and grown from birth on empty vector and gas-1 RNAi plates. At the L4 stage, C. elegans were transferred to plates with the drug compounds and TMRE, and co-exposed to the drug compounds and TMRE for 24 hours. The C. elegans L4 stage worms were then washed with 6 mL of S. basal, followed by incubation in S. basal for 30 min to clear residual fluorescence dye from their gastrointestinal tract. The COPAS Biosorter was used at excitation wavelength 561 nm and emission filter of 615 nm to measure the relative intensity of TMRE fluorescence in the worms' cells. The GFP fluorescence from C. elegans Cox-4::GFP were quantified utilizing excitation wavelength 488 nm and emission filter of 510 nm. To normalize the membrane potential intensity to mitochondrial mass intensity, red fluorescence was measured at an excitation wavelength 561 nm and emission filter at 625 nm.

In Vivo Efficacy Test in Mitochondrial Complex IV Disease Zebrafish Model—Larval Brain Death, Neuromuscular Tap/Touch Responses, and Heartbeat Presence

Efficacy tests were performed on wild-type (AB) and Surf1^{-/-} zebrafish, where SURF1 is a complex IV assembly factor. AB and Surf1^{-/-} zebrafish embryos were collected and sorted at 10 zebrafish per well at day 0. AB and Surf1^{-/-} zebrafish were then synchronized by bleaching and treated with pronase at 1 day post fertilization (dpf). AB and Surf1^{-/-} zebrafish at 5 dpf were pre-treated with the drug compounds (1 nM-10 μM). Following this pre-treatment, AB zebrafish at 6 dpf were co-treated with sodium azide (75 μM) to inhibit mitochondrial complex IV activity and the drug compounds. Alternatively, Surf1^{-/-} zebrafish at 6 dpf were co-treated with sodium azide (32.5 μM) and the drug compounds, then the phenotypes thereof were evaluated at 7 dpf. Specifically, AB and Surf1^{-/-} zebrafish were scored for effects of co-treatment of the drug compounds and azide compared to azide-only treated controls for physiologic parameters such as development of brain death, neuromuscular tap and touch responses, and heartbeat presence. Brain cell death indicative of a diseased state was recorded in zebrafish exhibiting gray discoloration in their brain region, which does not occur in healthy zebrafish larvae. Neuromuscular response was evaluated by touch response (when larvae were manually touched with a probe) and startle response (when the culture vessel was lightly tapped with a probe). Heartbeat was recorded as present or absent by microscopic visualization.

Efficacy Test in Mitochondrial Complex IV Disease Model—Swimming Activity Analysis

Swimming activity analysis was performed on AB zebrafish. Embryos and larvae were maintained at 28° C. Adult zebrafish were set pairwise in undivided mating tanks in order to collect and sort 10 zebrafish embryos per well. AB zebrafish were pre-treated with the drug compounds at 5 dpf, then co-treated with sodium azide (75 μM) and the drug compounds at 6 dpf. AB zebrafish were transferred on 7 dpf to 96-well plates, with 8 zebrafish per condition for swimming activity assay. The drug treatments were evaluated using the automated imaging system Zebra Box (ViewPoint Life Sciences) and data were collected using the ZebraLab software (ViewPoint Life Sciences). Zebrafish were acclimatized to 60% light for 10 min to achieve a stable level of activity, then exposed to 10 min of dark (0% light) to produce a startle response, in which locomotion increases to a maximum movement for the first 5 min and then decreases gradually to a stable level. This light cycling of 10 min of on and off was repeated 3 consecutive cycles per analysis to observe the rescue of the compound. The average of maximum movement of the zebrafish for the first 5 min of the dark cycles was analyzed in Graphpad Prism 7.04.

Results

Lifespan, Activity, and Growth In Vivo Assays in C. elegans—Trapidil and Metyrapone

Two drug compounds, trapidil and metyrapone were tested to validate their in vitro rescue effect in a dose-dependent manner (i.e., 1 nM, 10 nM, 100 nM, and 1 μM) on in vivo studies in the C. elegans gas-1(fc21) strain that harbors a mitochondrial complex I subunit (ndufs2^{-/-}) homozygous missense mutation that causes reduced mitochondrial complex I activity.

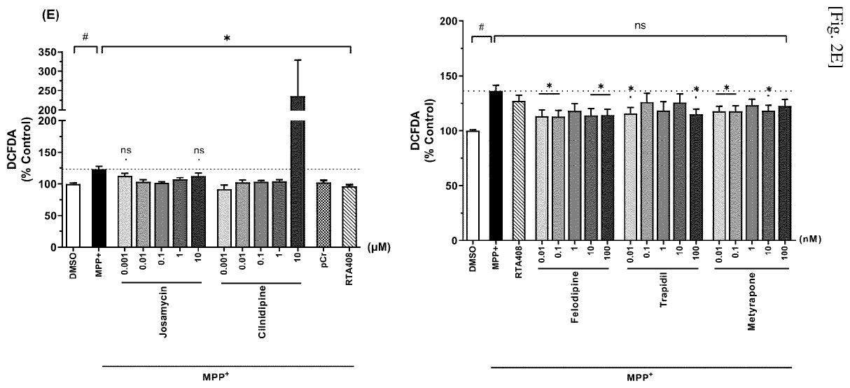

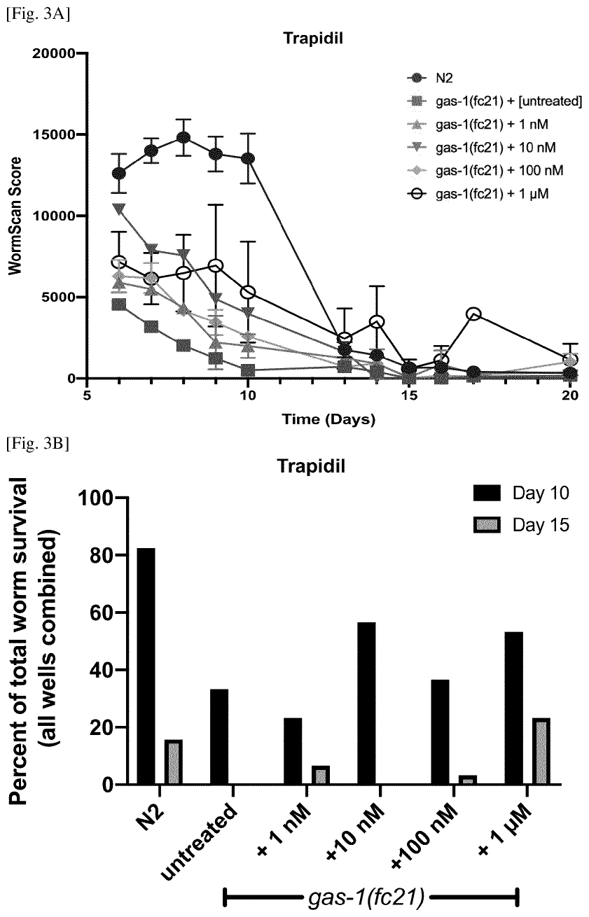

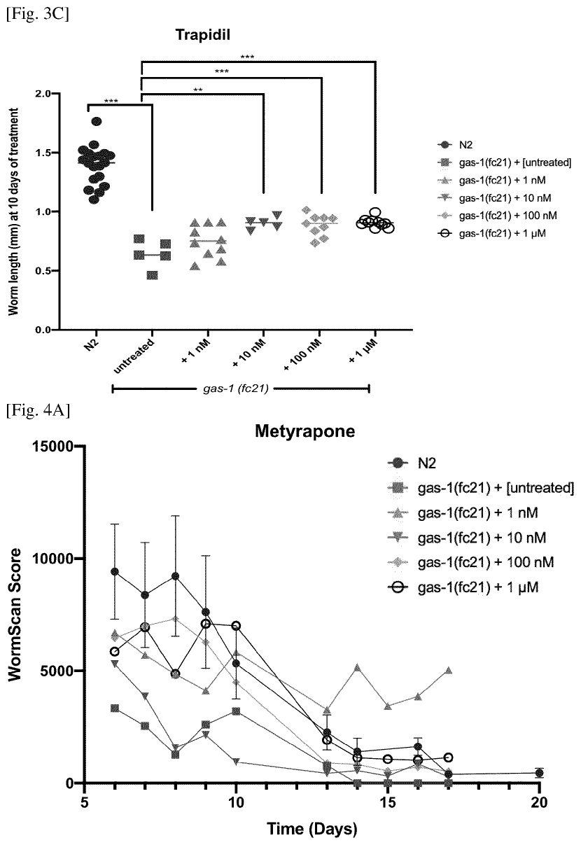

As shown in FIG. 3(A), the activity of C. elegans gas-1 was significantly reduced compared to wild-type (N2 Bristol) controls, while trapidil showed its beneficial effect to mitochondrial complex I mutant, gas-1(fc21) at the concentrations of 1 nM, 10 nM, 100 nM, and 1 μM. As shown in FIG. 3(B), the survival numbers of trapidil treated gas-1(fc21) animals were increased compared to untreated gas-1(fc21) animals at day 10 at the concentrations of 10 nM, 100 nM, and 1 μM, and at day 15 at the concentrations of 1 nM, 100 nM, and 1 μM. Further, as shown in FIG. 3(C), the body length of untreated gas-1(fc21) was significantly decreased relative to wild-type (N2 Bristol) controls, while it was gradually and significantly recovered as the concentration of trapidil increased from 1 nM to 1 μM.

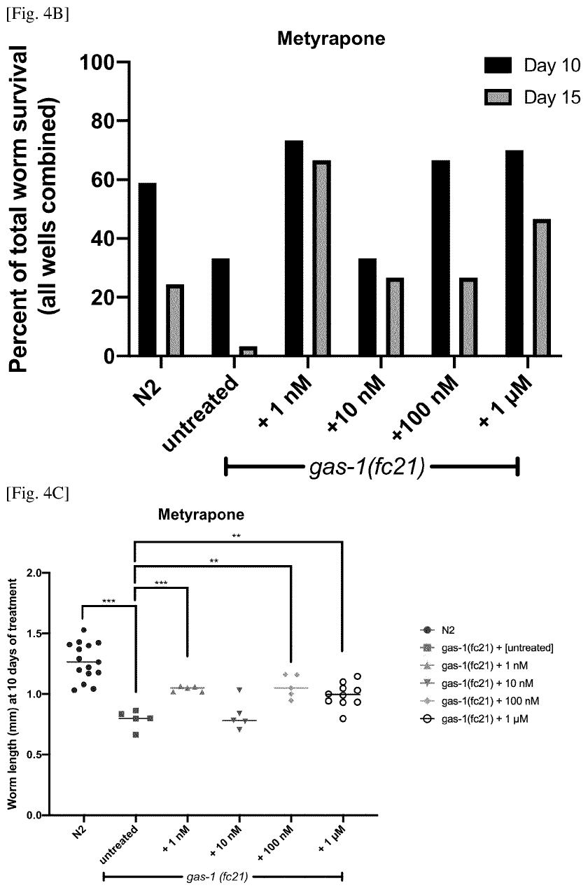

As shown in FIG. 4(A), metyrapone showed its beneficial effect in the mitochondrial complex I mutant gas-1(fc21) worms at the concentrations of 1 nM, 10 nM, 100 nM, and 1 μM in activity. As shown in FIG. 4(B), the survival numbers of metyrapone treated gas-1(fc21) worms were increased as compared to untreated gas-1(fc21) worms at days 10 and 15 at the concentrations of 1 nM, 10 nM, 100 nM, and 1 μM. Further, as shown in FIG. 4(C), the body length of gas-1(fc21) worms was significantly decreased relative to wild-type (N2 Bristol) controls, while it was recovered with statistically significant improvement at the concentrations of 1 nM, 100 nM, and 1 μM.

In Vivo Mitochondrial Stress Assay—Josamycin and Cilnidipine

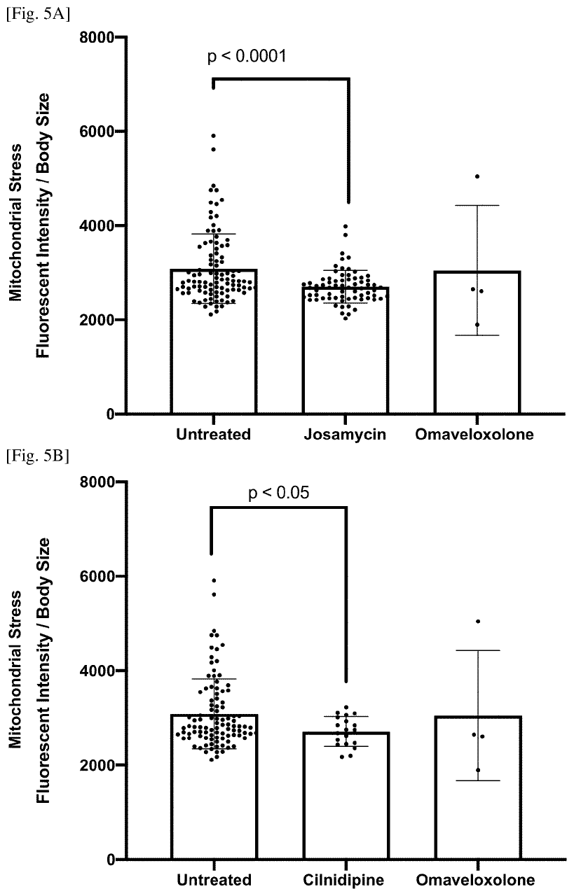

Josamycin and cilnidipine showed statistically significantly improved rescue effect on the high mitochondrial stress in the mitochondrial complex I mutant C. elegans strain gas-1(fc21). Specifically, C. elegans gas-1(fc21) worms that harbored the hsp-6p::GFP green fluorescence reporter were treated with buffer or drug (josamycin or cilnidipine) at 25 μM from L1 stage to L4 stages. Josamycin treated C. elegans gas-1(fc21) worms showed that their hsp-6p green fluorescence intensity was decreased by 12.48% as compared to untreated gas-1(fc21) worms, and cilnidipine treated gas-1(fc21) worms showed a 12.06% decreased hsp-6p green fluorescence intensity as compared to untreated gas-1(fc21) worms. These in vivo results demonstrate that josamycin and cilnidipine successfully ameliorated mitochondrial stress in a genetic-based complex I disease animal model (see FIGS. 5(A) and 5(B)).

TABLE 3

| | Average | % Reduction |
|---|---|---|
| C. elegans gas-1(fc21) | fluorescence | (compared to |
| worm Treatment | intensity | untreated gas-1 |
| Condition | (RFU) | (fc21)) |
| Untreated | 3085 | — |
| Josamycin treated | 2700 | 12.48% |
| Cilnidipine treated | 2713 | 12.06% |

In Vivo Mitochondrial Membrane Potential TMRE Assay—Cilnidipine

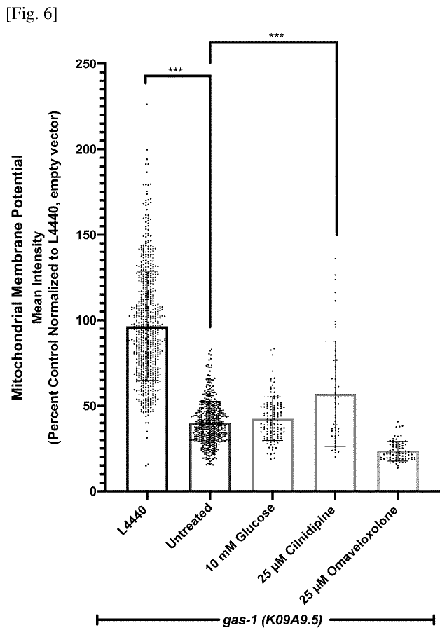

C. elegans worms used in the in vivo mitochondrial membrane potential TMRE assay were exposed to buffer or the drug compounds at 25 μM for 24 hours. As shown in FIG. 6, C. elegans gas-1 worms were studied by using feeding RNA interference (RNAi) to knockdown the K09A9.5 gene that encodes gas-1. Indeed, gas-1 RNAi worms showed significantly reduced mitochondrial membrane potential by 58.5% relative to L4440, an empty vector control with normal gas1 expression, showing the significant reduction in mitochondrial membrane potential that occurs in gas-1. Mitochondrial membrane potential in 25 μM cilnidipine treated gas-1 RNAi worms was statistically significantly increased by 42.3% comparing to untreated gas-1 RNAi worms (p<0.0001).

In Vivo Efficacy Test in Mitochondrial Complex IV Disease Zebrafish Model—Josamycin, Cyproterone, Felodipine, Trapidil and Metyrapone

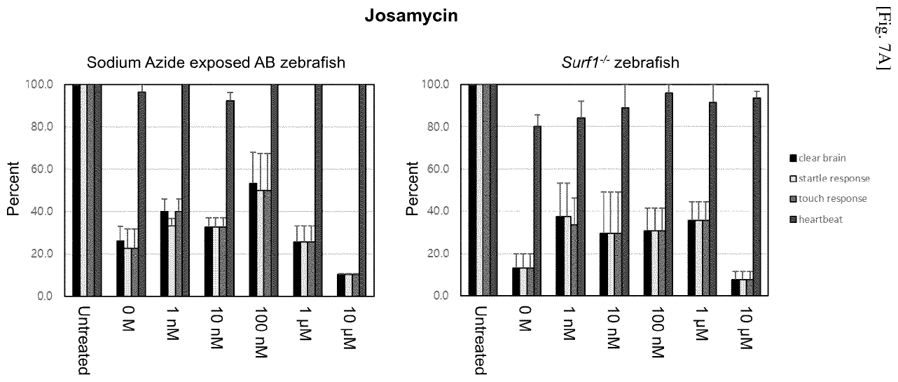

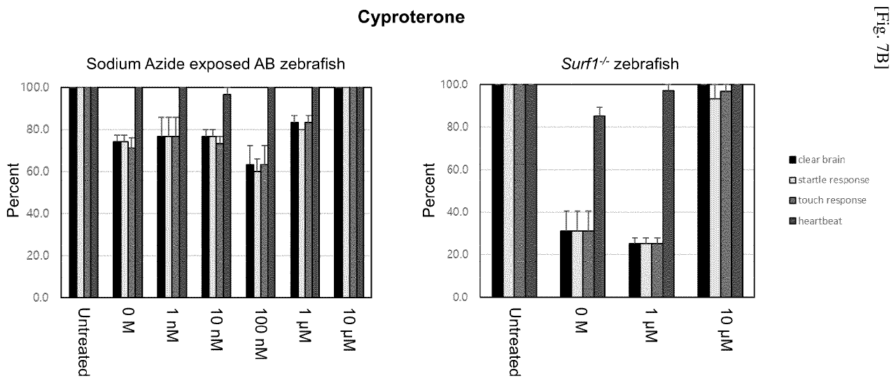

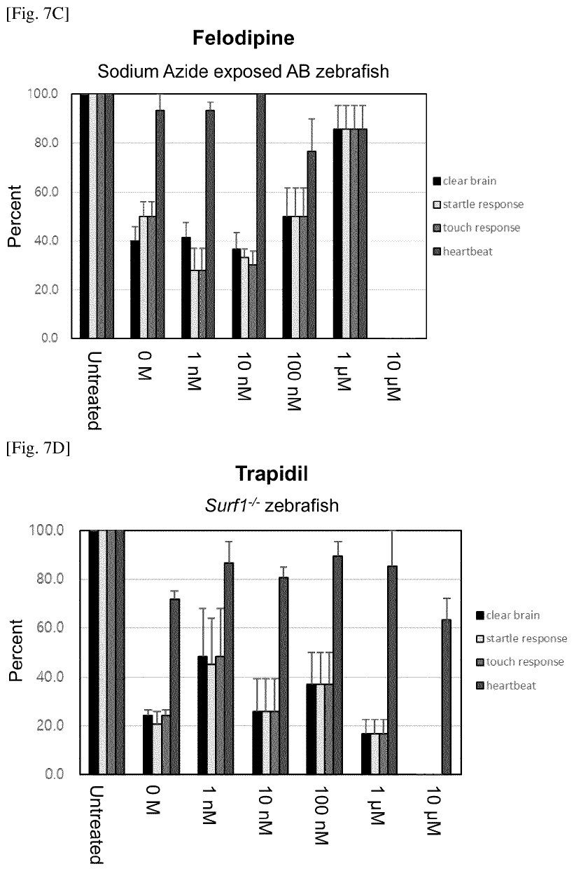

Josamycin was co-treated with sodium azide at 6 dpf in AB zebrafish or Surf1^{-/-} zebrafish, where SURF1 is a complex IV assembly factor whose deficiency is associated with human mitochondrial disease. As shown in FIG. 7(A), josamycin treated AB zebrafish showed prevention of 75 μM sodium azide-induced animal dysfunction (larval brain death, neuromuscular tap and touch responses, and heartbeat presence) at the concentrations of 1 nM through 100 nM. Further, josamycin treated Surf1^{-/-} zebrafish showed recovery of 32.5 μM sodium azide-induced dysfunction (larval brain death, neuromuscular tap and touch responses, and heartbeat presence) through the concentration of 1 nM to 1 μM.

As shown in FIG. 7(B), 10 μM of cyproterone acetate completely prevented sodium azide-induced animal dysfunction and death (larval brain death, neuromuscular responses, and heartbeat presence) in AB zebrafish treated with 75 μM of sodium azide. Further, 10 μM of cyproterone acetate prevented sodium azide-induced dysfunctions (larval brain death, neuromuscular tap and touch responses, and heartbeat presence) in Surf1^{-/-} zebrafish treated with 32.5 μM of sodium azide.

Felodipine treated AB zebrafish prevented sodium azide-induced animal dysfunction and death (larval brain death, neuromuscular tap and touch responses, and heartbeat presence) at the concentration of 100 nM through 1 μM (as shown in FIG. 7(C)). Surf1^{-/-} zebrafish were treated with trapidil across the concentration range of 1 nM to 10 μM. As shown in FIG. 7(D), trapidil yielded beneficial effect to prevent sodium azide-induced animal dysfunction and death (larval brain death, neuromuscular tap and touch responses, and heartbeat presence) at the concentrations of 1 nM through 100 nM. Metyrapone treated AB zebrafish prevented sodium azide-induced animal dysfunction (larval brain death, neuromuscular responses, and heartbeat presence) at the concentration of 1 μM (as shown in FIG. 7(E)).

In Vivo Swimming Activity Analysis in Mitochondrial Complex IV Disease Zebrafish Model—Cyproterone and Felodipine

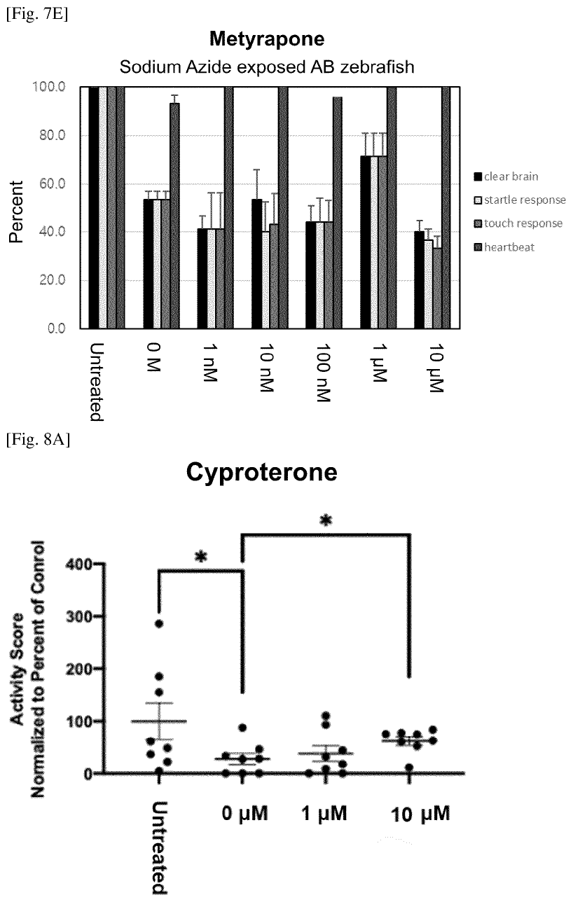

Sodium azide treated AB zebrafish showed decreased swimming activity by 50% as compared to untreated AB zebrafish. As shown in FIGS. 8(A) and 8(B), cyproterone acetate and felodipine treated AB zebrafish significantly rescued the swimming activity as compared to sodium azide treated AB zebrafish at the concentration of 10 μM and 500 nM, respectively (p<0.05).

### Example 4: ATP Content-Based Cell Viability Assay of Primary Mitochondrial Disease Human Subjects' Fibroblast Cell Lines to Validate AI-Discovered Drug Compounds

Method

Human Mitochondrial Disease Subjects' Fibroblast Cell Line In Vitro Viability Assay

Fibroblast cell lines (FCLs) were obtained from prior skin biopsies of human subjects performed in the Mitochondrial Medicine Clinic at The Children's Hospital of Philadelphia. FCLs were confirmed to be mycoplasma free. Specific cell lines studied included Q1881p1 for healthy control, Q1687F (NUBPL^{-/-}) for mitochondrial complex I deficiency disease, and Q1775F (SURF1^{-/-}) for mitochondrial complex IV deficiency disease. FCLs were grown in DMEM containing 1 g/L (5.5 mM) glucose and supplemented with 10% FBS, and 50 μg/mL uridine for 24 hours. Culture media was replaced with 10 mM galactose media supplemented with 10% FBS and 50 μg/mL uridine that did not contain any glucose as a means to stress mitochondrial deficiency cells, and then co-treated with the drug compounds for 72 hours. Fibroblast cell viability was evaluated using the CellTiter-Glo 2.0 Cell Viability Assay Kit (Promega) on one 96-well plate (n=4 wells/condition). Statistical analyses were performed by Student's T-test in Graphpad Prism.

Result

FCLs of human subjects (Q1687F or Q1775F) with mitochondrial complex I or IV deficiency disorders, respectively, had reduced cellular viability as indicated by intracellular ATP content that reflects their reduced mitochondrial oxidative phosphorylation capacity, when 10 mM galactose without glucose was used as an energy source, since ATP-generating function relies solely on mitochondrial oxidative phosphorylation function without contribution of anaerobic glycolysis when only galactose is present.

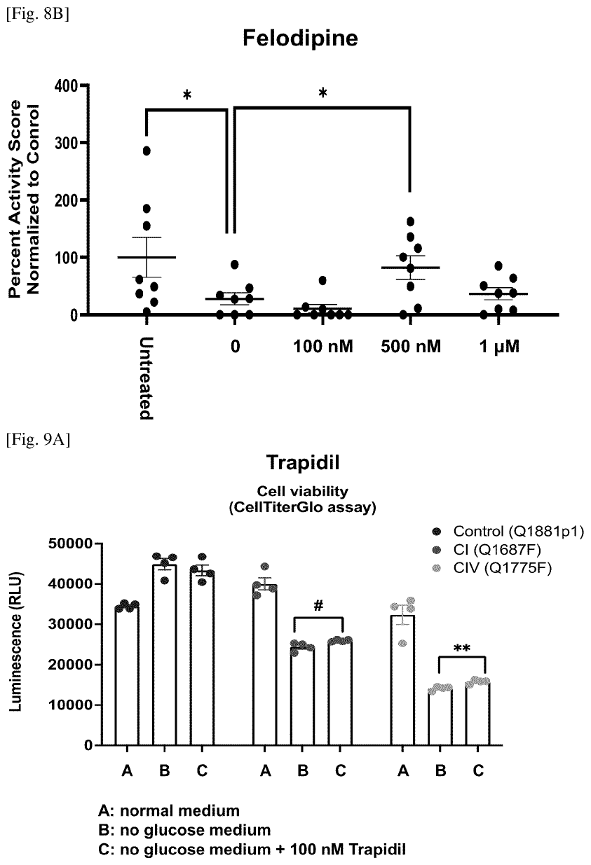

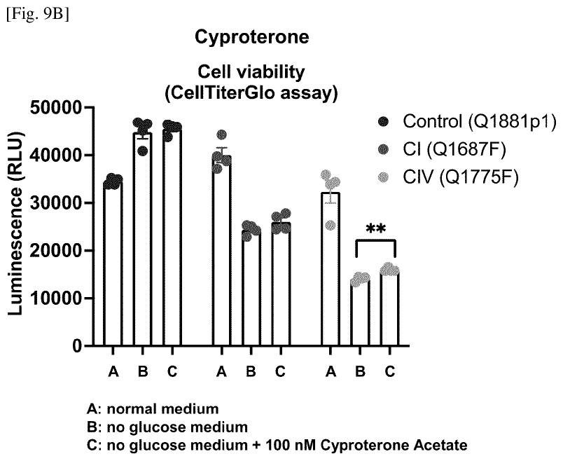

As shown in FIGS. 9(A) and (B), FCLs from a human subject (Q1775F) with mitochondrial complex IV deficiency had 11.6% (p=0.004) and 13.2% (p=0.001) statistically significant improvement in cell viability (based on total ATP levels) upon treatment with 100 nM of trapidil and cyproterone acetate, respectively. In addition, 100 nM of trapidil had marginally statistically significant efficacy, with 6.5% (p=0.058) improvement in cell viability in FCL from a human subject (Q1687F) with a severe mitochondrial complex I deficiency disorder.

Summary

The inventors validated the efficacy of the six (6) drug compounds that were selected though Standigm's AI technology to ameliorate mitochondrial function that was impaired by pharmacologic toxins by employing an MPP⁺-exposed SH-SY5Y human cell model. In the nanomolar range of treatment, the six drug compounds showed the increased survival ability and the ameliorated mitochondrial activities such as enhanced cell metabolic viability, increased intracellular ATP content, increased mitochondrial membrane potential, and reduced mitochondrial or intracellular ROS. The expected efficacy for mitochondrial disease was also validated by employing in vivo animal studies using both pharmacologic stressors (e.g., sodium azide) and genetic models such as C. elegans gas-1(fc21) and D. rerio Surf1^{-/-}, and finally human mitochondrial disease subjects' primary fibroblast cell lines. Josamycin, cilnidipine, trapidil and metyrapone showed significant beneficial effects on C. elegans gas-1(fc21) and gas-1 RNA interference knockdown mitochondrial complex I disease models at the level of lifespan, swimming activity, growth, and diverse aspects of in vivo mitochondrial functions including mitochondrial membrane potential and mitochondrial stress. Mitochondrial complex IV sodium azide inhibited or Surf1^{-/-} genetic mutant zebrafish showed significantly improved swimming activity, prevention of brain death, neuromuscular functions, and preservation of heartbeat when treated with josamycin, cyproterone, felodipine, trapidil or metyrapone. Surprisingly, cyproterone and trapidil showed statistically significantly beneficial effects on survival under prolonged metabolic stress of a human mitochondrial complex IV disease subject's primary fibroblast cell line, indicating their promising therapeutic efficacy to patients with mitochondrial diseases. Collectively, these six marketed drug compounds whose efficacy was cross-validated in mitochondrial diseases models from at least two different evolutionarily-distinct species would be beneficial in human mitochondrial disease, for whom no FDA approved effective therapies currently exist. These results indicate that the active compounds discussed herein will be useful to treat human mitochondrial disease patients and/or enhance in vivo mitochondrial function in cells and tissues of mitochondrial disease patients.

Although the presently disclosed subject matter and its advantages have been described in detail, it should be understood that various changes, substitutions and alterations can be made herein without departing from the spirit and scope of the invention. Moreover, the scope of the present application is not intended to be limited to the particular embodiments of the process, machine, manufacture, and composition of matter, means, methods and steps described in the specification. As one of ordinary skill in the art will readily appreciate from the invention of the presently disclosed subject matter, processes, machines, manufacture, compositions of matter, means, methods, or steps, presently existing or later to be developed that perform substantially the same function or achieve substantially the same results as the corresponding embodiments described herein may be utilized according to the presently disclosed subject matter. Accordingly, the appended claims are intended to include within their scope such process, machines, manufacture, compositions of matter, means, methods, or steps. Various patents, patent application, publications, product descriptions, protocols, and sequence accession numbers are cited throughout this application, the inventions of which are incorporated herein by reference in their entireties for all purposes.

What is claimed is:

1. A method for enhancing mitochondrial function in a subject in need thereof, comprising administering to the subject an effective amount of one or more compounds selected from the group consisting of: josamycin, cyproterone, cilnidipine, felodipine, trapidil, metyrapone, and a pharmaceutically acceptable salt thereof.

2. The method according to claim 1, wherein a pharmaceutically acceptable salt of cyproterone is acetate.

3. The method according to claim 1, wherein enhancing mitochondrial function is one or more events selected from the group consisting of:
(i) enhancing cell viability;
(ii) increasing intracellular ATP content;
(iii) increasing mitochondrial membrane potential;
(iv) reducing mitochondrial reactive oxygen species (ROS);
(v) reducing intracellular ROS; and
(vi) reducing mitochondrial stress,
compared to prior to the administration of the one or more compounds.

4. The method according to claim 1, wherein enhancing mitochondrial function is one or more events selected from the group consisting of:
(i) increasing lifespan;
(ii) enhancing neuronal activity;
(iii) enhancing locomotor activity; and
(iv) enhancing growth,
compared to prior to the administration of the one or more compounds.

5. A method for treating a mitochondrial disease by enhancing mitochondrial function in a subject in need thereof, comprising administering to such subject an effective amount of one or more compounds selected from the group consisting of:
josamycin, cyproterone, cilnidipine, felodipine, trapidil, metyrapone, and a pharmaceutically acceptable salt thereof.

6. The method according to claim 5, a pharmaceutically acceptable salt of cyproterone is acetate.

7. The method according to claim 5, wherein the mitochondrial disease comprises one or more selected from the group consisting of:
Leber hereditary optic neuropathy (LHON), mitochondrial myopathy, encephalopathy, lactic acidosis and stroke-like episodes (MELAS) syndrome, mitochondrial complex I deficiency, mitochondrial complex II deficiency, mitochondrial complex III deficiency, mitochondrial complex IV deficiency, mitochondrial complex V deficiency, Leigh syndrome, autosomal dominant optic atrophy (ADOA), leukoencephalopathy with brain stem and spinal cord involvement and lactate elevation (LBSL), Luft disease, multiple acyl-CoA dehydrogenase (MAD) deficiency, mitochondrial enoyl CoA reductase protein-associated neurodegeneration (MEPAN) syndrome, mitochondrial DNA depletion, mitochondrial encephalopathy, pyruvate carboxylase deficiency, mitochondrial myopathy, Friedreich's ataxia, Barth syndrome, fatal infantile cardioencephalomyopathy, Charcot-Marie-Tooth disease, infantile lactic acidosis, congenital lactic acidosis (CLA), chronic lactic acidosis, Kearns-Sayre syndrome (KSS), mitochondrially inherited diabetes and deafness (MIDD), Alpers-Huttenlocher syndrome (AHS), childhood myocerebrohepatopathy spectrum (MCHS), ataxia neuropathy spectrum (ANS; previously referred to as mitochondrial recessive ataxia syndrome (MIRAS) and sensory ataxia neuropathy dysarthria and ophthalmoplegia (SANDO)), myoclonic epilepsy myopathy sensory ataxia (MEMSA; previously referred to as spinocerebellar ataxia with epilepsy (SCAE)), Sengers syndrome, MEGDEL syndrome (also known as 3-methylglutaconic aciduria with deafness, encephalopathy and Leigh-like syndrome), Pearson syndrome, myoclonic epilepsy with ragged red fibers (MERRF), neurogenic muscle weakness, ataxia and retinitis pigmentosa (NARP), chronic progressive external ophthalmoplegia (CPEO), mitochondrial neurogastrointestinal encephalopathy (MNGIE) syndrome, carnitine deficiency, carnitine-acylcarnitine translocase (CACT) deficiency, carnitine palm itoyl transferase 1A (CPT I) deficiency, carnitine palmitoyl transferase (CPT II) deficiency, creatine deficiency syndromes, creatine deficiency syndromes which contain guanidinoacetate methyltransferase (GAMT) deficiency, L-arginine:glycine amidinotransferase (AGAT) deficiency, or creatine transporter deficiency (including SLC6A8-related creatine transporter deficiency), thymidine kinase 2 deficiency (TK2D), pyruvate dehydrogenase complex deficiency (PDCD), fatty acid oxidation disorders (FAOD), fatty acid oxidation disorders which contain acyl-CoA dehydrogenase 9 (ACAD9) deficiency, multiple acyl-CoA dehydrogenase deficiency (MADD), long-chain acyl-CoA dehydrogenase (LCAD) deficiency, long-chain 3-hydroxyacyl-CoA dehydrogenase (LCHAD) deficiency, medium-chain acyl-CoA dehydrogenase (MCAD) deficiency, short-chain acyl-CoA dehydrogenase (SCAD) deficiency, short-chain 3-hydroxyacyl-CoA dehydrogenase (SCHAD) deficiency or very long-chain acyl-CoA dehydrogenase (VLCAD) deficiency, co-enzyme Q10 deficiency, and multiple mitochondrial dysfunction syndrome.

8. A composition for enhancing mitochondrial function in a subject in need thereof and/or for treating a mitochondrial disease by enhancing mitochondrial function in a subject in need thereof, said composition comprising, as an active ingredient, one or more compounds selected from the group consisting of josamycin, cyproterone, cilnidipine, felodipine, trapidil, metyrapone, and a pharmaceutically acceptable salt thereof, wherein an effective amount of the composition is administered to the subject.

9. The composition according to claim 8, wherein the active ingredient is cyproterone acetate.

10. The composition according to claim 8, wherein enhancing mitochondrial function is one or more events selected from the group consisting of:
(i) enhancing cell viability;
(ii) increasing intracellular ATP content;
(iii) increasing mitochondrial membrane potential;
(iv) reducing mitochondrial reactive oxygen species (ROS);
(v) reducing intracellular ROS; and
(vi) reducing mitochondrial stress,
compared to prior to the administering of the one or more compounds.

11. The composition according to claim 8, wherein enhancing mitochondrial function is one or more events selected from the group consisting of:
(i) increasing lifespan;
(ii) enhancing neuronal activity;
(iii) enhancing locomotor activity; and
(iv) enhancing growth,
compared to prior to the administering of the one or more compounds.

12. The composition according to claim 8, wherein the mitochondrial disease comprises one or more selected from the group consisting of:
Leber hereditary optic neuropathy (LHON), mitochondrial myopathy, encephalopathy, lactic acidosis and stroke-like episodes (MELAS) syndrome, mitochondrial complex I deficiency, mitochondrial complex II deficiency, mitochondrial complex III deficiency, mitochondrial complex IV deficiency, mitochondrial complex V deficiency, Leigh syndrome, autosomal dominant optic atrophy (ADOA), leukoencephalopathy with brain stem and spinal cord involvement and lactate elevation (LBSL), Luft disease, multiple acyl-CoA dehydrogenase (MAD) deficiency, mitochondrial enoyl CoA reductase protein-associated neurodegeneration (MEPAN) syndrome, mitochondrial DNA depletion, mitochondrial encephalopathy, pyruvate carboxylase deficiency, mitochondrial myopathy, Friedreich's ataxia, Barth syndrome, fatal infantile cardioencephalomyopathy, Charcot-Marie-Tooth disease, infantile lactic acidosis, congenital lactic acidosis (CLA), chronic lactic acidosis, Kearns-Sayre syndrome (KSS), mitochondrially inherited diabetes and deafness (MIDD), Alpers-Huttenlocher syndrome (AHS), childhood myocerebrohepatopathy spectrum (MCHS), ataxia neuropathy spectrum (ANS; previously referred to as mitochondrial recessive ataxia syndrome (MIRAS) and sensory ataxia neuropathy dysarthria and ophthalmoplegia (SANDO)), myoclonic epilepsy myopathy sensory ataxia (MEMSA; previously referred to as spinocerebellar ataxia with epilepsy (SCAE)), Sengers syndrome, MEGDEL syndrome (also known as 3-methylglutaconic aciduria with deafness, encephalopathy and Leigh-like syndrome), Pearson syndrome, myoclonic epilepsy with ragged red fibers (MERRF), neurogenic muscle weakness, ataxia and retinitis pigmentosa (NARP), chronic progressive external ophthalmoplegia (CPEO), mitochondrial neurogastrointestinal encephalopathy (MNGIE) syndrome, carnitine deficiency, carnitine-acylcarnitine translocase (CACT) deficiency, carnitine palm itoyl transferase 1A (CPT I) deficiency, carnitine palmitoyl transferase (CPT II) deficiency, creatine deficiency syndromes, creatine deficiency syndromes which contain guanidinoacetate methyltransferase (GAMT) deficiency, L-arginine:glycine amidinotransferase (AGAT) deficiency, or creatine transporter deficiency (including SLC6A8-related creatine transporter deficiency), thymidine kinase 2 deficiency (TK2D), pyruvate dehydrogenase complex deficiency (PDCD), fatty acid oxidation disorders (FAOD), fatty acid oxidation disorders which contain acyl-CoA dehydrogenase 9 (ACAD9) deficiency, multiple acyl-CoA dehydrogenase deficiency (MADD), long-chain acyl-CoA dehydrogenase (LCAD) deficiency, long-chain 3-hydroxyacyl-CoA dehydrogenase (LCHAD) deficiency, medium-chain acyl-CoA dehydrogenase (MCAD) deficiency, short-chain acyl-CoA dehydrogenase (SCAD) deficiency, short-chain 3-hydroxyacyl-CoA dehydrogenase (SCHAD) deficiency or very long-chain acyl-CoA dehydrogenase (VLCAD) deficiency, co-enzyme Q10 deficiency, and multiple mitochondrial dysfunction syndrome.
